(19) United States
(12) Patent Application Publication
(10) Pub. No.: US 2023/0053060 A1
(43) Pub. Date: Feb. 16, 2023

(54) METHOD FOR MANUFACTURING INDUCED PLURIPOTENT STEM CELLS

(71) Applicants: Koji TANABE, Palo Alto, CA (US); I Peace, Inc., Palo Alto, CA (US)

(72) Inventors: Koji TANABE, Palo Alto, CA (US); Kenta SUTO, Palo Alto, CA (US)

(73) Assignee: I Peace, Inc., Palo Alto, CA (US)

(21) Appl. No.: 17/759,308

(22) PCT Filed: Jan. 22, 2021
(86) PCT No.: PCT/JP2021/002335

§ 371 Date: Jul. 22, 2022

Related U.S. Application Data
(60) Provisional application No. 63/131,451, filed on Dec. 29, 2020.
(60) Provisional application No. 62/965,669, filed on Jan. 24, 2020.

(51) Int. Cl. C12N 15/88; C12N 15/86; C12N 5/0735

(52) U.S. Cl. CPC C12N 15/88; C12N 15/86; C12N 5/0606

(57) ABSTRACT

According to the present disclosure, there is provided a method for manufacturing pluripotent stem cells including introducing a reprogramming factor into cells and seeding the cells, into which the reprogramming factor is introduced, at a low concentration and culturing the cells.

### TECHNICAL FIELD

The present invention relates to a cell technology and a method for manufacturing induced pluripotent stem cells.

### BACKGROUND ART

Induced pluripotent stem (iPS) cells are cells having two characteristic abilities. One is an ability to transform into all the cells that constitute a body. The other is to have a semi-permanent proliferative ability. Since iPS cells have these two abilities, the iPS cells can be applied to a transplantation treatment without being rejected by manufacturing iPS cells from their own somatic cells and transforming them into target somatic cells. Therefore, iPS cells are considered to be a key technology for regenerative medicine.

From the creation of iPS cells to the present, many methods for manufacturing iPS cells have been established. Examples of typical methods for manufacturing iPS cells include a method using retroviruses/lentiviruses and a method using an episomal vector.

The method using retroviruses/lentiviruses will be described. Somatic cells are infected with retroviruses or lentiviruses, and genes encoding a reprogramming factor can be introduced into cells. In addition, retroviruses or lentiviruses allow the reprogramming factor to be inserted into the genome of somatic cells and induce stable expression of the reprogramming factor in the cells.

However, the method using retroviruses/lentiviruses has the following problems. First, insertion of the reprogramming factor into the genome of somatic cells damages existing genes and promoters, which can cause canceration of the cells. In addition, the reprogramming factor inserted into the genome may be reactivated after iPS cells are transformed to somatic cells. Therefore, cells for transplantation derived from iPS cells have a risk of canceration. Actually, in a mouse model, reactivation of the introduced reprogramming factor is observed in somatic cells, and canceration has been confirmed (for example, refer to Non-Patent Document 1).

An episomal vector is circular DNA and self-amplified in the nucleus. It had been thought that an episomal vector is not integrated into the genome, in principle, but in recent studies, it has been reported that fragments of episomal vectors are scattered and inserted into the genome of iPS cells created by episomes. Therefore, there is a problem of the reprogramming gene remaining in the cells. For example, if c-MYC or KLF4 remains in the cells, it causes canceration. It is extremely expensive to examine whether the reprogramming gene remains in cells. It is not possible to show that all cells in the transplanted cell pool have no insertion of the episomal plasmid into the genome and have no residue.

Since the method using retroviruses/lentiviruses and the method using an episomal vector have the above problems, methods for manufacturing iPS cells using RNA have been proposed (for example, refer to Patent Document 1 to 4 and Non-Patent Document 2).

### CITATION LIST

### Patent Document

Patent Document 1: WO 2000/70070
Patent Document 2: WO 2010/008054
Patent Document 3: WO 2012/029770
Patent Document 4: WO 2015/046229

### Non-Patent Document

Non-Patent Document 1: Nature 448, 313-317
Non-Patent Document 2: Nature Biotechnol 26(3): 313-315, 2008.

### SUMMARY

### Technical Problem

There is a demand for not only a method for introducing a reprogramming factor but also a method for efficiently introducing a reprogramming factor into somatic cells. One object of the present invention is to provide a method for efficiently manufacturing induced pluripotent stem cells.

### Solution to Problem

According to an aspect of the present invention, there is provided a method for manufacturing pluripotent stem cells including introducing a reprogramming factor into cells and seeding the cells, into which the reprogramming factor is introduced, at a low concentration and culturing the cells.

In the method for manufacturing pluripotent stem cells, the low concentration may be 0.25×10⁴ cells/cm² or less.

In the method for manufacturing pluripotent stem cells, the low concentration may be a concentration at which 11 or more of the seeded cells do not come into contact with each other.

In the method for manufacturing pluripotent stem cells, the low concentration may be 5% or less confluency.

In the method for manufacturing pluripotent stem cells, the reprogramming factor may be RNA.

In the method for manufacturing pluripotent stem cells, the reprogramming factor may be introduced into the cells by a lipofection method.

In the method for manufacturing pluripotent stem cells, the reprogramming factor may be introduced into the cells by using a viral vector.

In the method for manufacturing pluripotent stem cells, the viral vector may be an RNA viral vector.

In the method for manufacturing pluripotent stem cells, the RNA viral vector may be a Sendai viral vector.

In the method for manufacturing pluripotent stem cells, the viral vector may be a temperature-sensitive viral vector in which stability of a viral nucleic acid decreases at a predetermined temperature or higher.

In the method for manufacturing pluripotent stem cells, the viral vector may not include a viral vector in which the stability of a viral nucleic acid does not decrease at the predetermined temperature or higher.

In the method for manufacturing pluripotent stem cells, the temperature-sensitive viral vector may be a Sendai viral vector into which at least one temperature-sensitive mutation selected from among TS7, TS12, TS13, TS14, and TS15 is introduced.

In the method for manufacturing pluripotent stem cells, the viral vector may not include a viral vector having lower temperature sensitivity than the temperature-sensitive Sendai viral vector into which the temperature-sensitive mutation selected from among TS7, TS12, TS13, TS14, and TS15 is introduced.

In the method for manufacturing pluripotent stem cells, the viral vector may include a viral vector having a temperature sensitivity equal to or higher than that of the temperature-sensitive Sendai viral vector into which the temperature-sensitive mutation selected from among TS7, TS12, TS13, TS14, and TS15 is introduced.

In the method for manufacturing pluripotent stem cells, after the reprogramming factor is introduced, the cells may be cultured at a temperature lower than the predetermined temperature for at least two days.

In the method for manufacturing pluripotent stem cells, after at least two days, the cells may be cultured at the predetermined temperature or higher.

In the method for manufacturing pluripotent stem cells, in the culturing, the cells into which the reprogramming factor is introduced may be passaged and cultured at the predetermined temperature or higher.

In the method for manufacturing pluripotent stem cells, passage in which cells are seeded at a low concentration may be repeated.

In the method for manufacturing pluripotent stem cells, passage in which cells are seeded at a low concentration of 0.25×10⁴ cells/cm² or less may be repeated.

In the method for manufacturing pluripotent stem cells, passage in which cells are seeded at a low concentration at which 11 or more of the seeded cells do not come into contact with each other may be repeated.

In the method for manufacturing pluripotent stem cells, in the culturing, pluripotent stem cells or colonies of pluripotent stem cells may not be isolated.

In the method for manufacturing pluripotent stem cells, the culturing may be adherent culture.

In the method for manufacturing pluripotent stem cells, the culturing may be suspension-culture.

In the method for manufacturing pluripotent stem cells, in the culturing, the cells into which the reprogramming factor is introduced may be cultured without cloning.

In the method for manufacturing pluripotent stem cells, in the culturing, the cells into which the reprogramming factor is introduced may be separated from an incubator, and at least some of the separated cells may be mixed and seeded. The separated cells may be mixed.

In the method for manufacturing pluripotent stem cells, in the culturing, the cells into which the reprogramming factor is introduced may be recovered from an incubator, and at least some of the recovered cells may be mixed and seeded. The recovered cells may be mixed.

In the method for manufacturing pluripotent stem cells, in the culturing, each of a plurality of colonies formed by the cells into which the reprogramming factor is introduced may not be picked up.

In the method for manufacturing pluripotent stem cells, in the culturing, cells derived from different single cells, which are the cells into which the reprogramming factor is introduced, may be mixed and seeded.

In the method for manufacturing pluripotent stem cells, in the passage, the cells into which the reprogramming factor is introduced may be mixed with each other.

In the method for manufacturing pluripotent stem cells, in the passage, clones of the cells into which the reprogramming factor is introduced may be mixed with each other.

In the method for manufacturing pluripotent stem cells, in the passage, the cells may not be cloned.

The method for manufacturing pluripotent stem cells may not include isolating the plurality of colonies formed by the cells into which the reprogramming factor is introduced.

In the method for manufacturing pluripotent stem cells, the plurality of colonies formed by the cells into which the reprogramming factor is introduced may be mixed with each other.

The method for manufacturing pluripotent stem cells may not include cloning a single colony formed by the cells into which the reprogramming factor is introduced.

The method for manufacturing pluripotent stem cells may not include picking up the colonies formed by the cells into which the reprogramming factor is introduced.

In the method for manufacturing pluripotent stem cells, cells attached to the incubator, which are the cells into which the reprogramming factor is introduced, may be recovered and at least some of the recovered cells may be seeded in a medium.

In the method for manufacturing pluripotent stem cells, the cells into which the reprogramming factor is introduced may be passaged without distinguishing them according to a gene expression state.

In the method for manufacturing pluripotent stem cells, the cells into which the reprogramming factor is introduced may be passaged without distinguishing them according to the degree of reprogramming.

In the method for manufacturing pluripotent stem cells, after the culturing, all cells may be cryopreserved as pluripotent stem cells.

In the method for manufacturing pluripotent stem cells, after the culturing, all separated cells may be cryopreserved as pluripotent stem cells.

In the method for manufacturing pluripotent stem cells, after the culturing, all cells separated with a separation solution may be cryopreserved as pluripotent stem cells.

In the method for manufacturing pluripotent stem cells, after the culturing, colonies of pluripotent stem cells may be mixed with each other.

In addition, according to an aspect of the present invention, there is provided a method for inducing pluripotent stem cells and eliminating a viral vector, the method including introducing a reprogramming factor into cells by using a viral vector; and seeding the cells, into which the reprogramming factor is introduced, at a low concentration and culturing the cells.

In the method for inducing pluripotent stem cells and eliminating a viral vector, the low concentration may be 0.25×10⁴ cells/cm² or less.

In the method for inducing pluripotent stem cells and eliminating a viral vector, the low concentration may be a concentration at which 11 or more of the seeded cells do not come into contact with each other.

In the method for inducing pluripotent stem cells and eliminating a viral vector, the low concentration may be or less confluency.

In the method for inducing pluripotent stem cells and eliminating a viral vector, the reprogramming factor may be RNA.

In the method for inducing pluripotent stem cells and eliminating a viral vector, the reprogramming factor may be introduced into the cells by a lipofection method.

In the method for inducing pluripotent stem cells and eliminating a viral vector, the reprogramming factor may be introduced into the cells using a viral vector.

In the method for inducing pluripotent stem cells and eliminating a viral vector, the viral vector may be an RNA viral vector.

In the method for inducing pluripotent stem cells and eliminating a viral vector, the RNA viral vector may be a Sendai viral vector.

In the method for inducing pluripotent stem cells and eliminating a viral vector, the viral vector may be a temperature-sensitive viral vector in which the stability of a viral nucleic acid decreases at a predetermined temperature or higher.

In the method for inducing pluripotent stem cells and eliminating a viral vector, the viral vector may not include a viral vector in which the stability of a viral nucleic acid does not decrease at the predetermined temperature or higher.

In the method for inducing pluripotent stem cells and eliminating a viral vector, the temperature-sensitive viral vector may be a Sendai viral vector into which at least one temperature-sensitive mutation selected from among TS7, TS12, TS13, TS14, and TS15 is introduced.

In the method for inducing pluripotent stem cells and eliminating a viral vector, the viral vector may not include a viral vector having lower temperature sensitivity than the temperature-sensitive Sendai viral vector into which the temperature-sensitive mutation selected from among TS7, TS12, TS13, TS14, and TS15 is introduced.

In the method for inducing pluripotent stem cells and eliminating a viral vector, the viral vector may not include a viral vector having a temperature sensitivity equal to or higher than that of the temperature-sensitive Sendai viral vector into which the temperature-sensitive mutation selected from among TS7, TS12, TS13, TS14, and TS15 is introduced.

In the method for inducing pluripotent stem cells and eliminating a viral vector, after the reprogramming factor is introduced, the cells may be cultured at a temperature lower than the predetermined temperature for at least two days.

In the method for inducing pluripotent stem cells and eliminating a viral vector, after at least two days, the cells may be cultured at the predetermined temperature or higher.

In the method for inducing pluripotent stem cells and eliminating a viral vector, in the culturing, the cells into which the reprogramming factor is introduced may be passaged and cultured at the predetermined temperature or higher.

In the method for inducing pluripotent stem cells and eliminating a viral vector, passage in which cells are seeded at a low concentration may be repeated.

In the method for inducing pluripotent stem cells and eliminating a viral vector, passage in which cells are seeded at a low concentration of 0.25×10⁴ cells/cm² or less may be repeated.

In the method for inducing pluripotent stem cells and eliminating a viral vector, passage in which cells are seeded at a low concentration at which 11 or more of the seeded cells do not come into contact with each other may be repeated.

In the method for inducing pluripotent stem cells and eliminating a viral vector, in the culturing, colonies of pluripotent stem cells or pluripotent stem cells may not be isolated.

In the method for inducing pluripotent stem cells and eliminating a viral vector, the culturing may be adherent culture.

In the method for inducing pluripotent stem cells and eliminating a viral vector, the culturing may be suspension-culture.

In the method for inducing pluripotent stem cells and eliminating a viral vector, in the culturing, culture may be performed without cloning the reprogramming factor.

In the method for inducing pluripotent stem cells and eliminating a viral vector, in the culturing, the cells into which the reprogramming factor is introduced may be separated from an incubator, and at least some of the separated cells may be mixed and seeded. The separated cells may be mixed.

In the method for inducing pluripotent stem cells and eliminating a viral vector, in the culturing, the cells into which the reprogramming factor is introduced may be recovered from an incubator, and at least some of the recovered cells may be mixed and seeded. The recovered cells may be mixed.

In the method for inducing pluripotent stem cells and eliminating a viral vector, in the culturing, each of a plurality of colonies formed by the cells into which the reprogramming factor is introduced may not be picked up.

In the method for inducing pluripotent stem cells and eliminating a viral vector, in the culturing, cells derived from different single cells, which are the cells into which the reprogramming factor is introduced, may be mixed and seeded.

In the method for inducing pluripotent stem cells and eliminating a viral vector, in the passage, the cells into which the reprogramming factor is introduced may be mixed with each other.

In the method for inducing pluripotent stem cells and eliminating a viral vector, in the passage, clones of the cells into which the reprogramming factor is introduced may be mixed with each other.

In the method for inducing pluripotent stem cells and eliminating a viral vector, in the passage, the cells may not be cloned.

The method for inducing pluripotent stem cells and eliminating a viral vector may not include isolating the plurality of colonies formed by the cells into which the reprogramming factor is introduced.

In the method for inducing pluripotent stem cells and eliminating a viral vector, the plurality of colonies formed by the cells into which the reprogramming factor is introduced may be mixed with each other.

The method for inducing pluripotent stem cells and eliminating a viral vector may not include cloning a single colony formed by the cells into which the reprogramming factor is introduced.

The method for inducing pluripotent stem cells and eliminating a viral vector may not include picking up the colonies formed by the cells into which the reprogramming factor is introduced.

In the method for inducing pluripotent stem cells and eliminating a viral vector, cells attached to the incubator, which are the cells into which the reprogramming factor is introduced, may be recovered and at least some of the recovered cells may be seeded in a medium.

In the method for inducing pluripotent stem cells and eliminating a viral vector, the cells into which the reprogramming factor is introduced may be passaged without distinguishing them according to a gene expression state.

In the method for inducing pluripotent stem cells and eliminating a viral vector, the cells into which the reprogramming factor is introduced may be passaged without distinguishing them according to the degree of reprogramming.

In the method for inducing pluripotent stem cells and eliminating a viral vector, after the culturing, all cells may be cryopreserved as pluripotent stem cells.

In the method for inducing pluripotent stem cells and eliminating a viral vector, after the culturing, all separated cells may be cryopreserved as pluripotent stem cells.

In the method for inducing pluripotent stem cells and eliminating a viral vector, after the culturing, all cells separated with a separation solution may be cryopreserved as pluripotent stem cells.

In the method for inducing pluripotent stem cells and eliminating a viral vector, after the culturing, all cells may be cryopreserved as pluripotent stem cells.

In the method for inducing pluripotent stem cells and eliminating a viral vector, after the culturing, colonies of pluripotent stem cells may be mixed with each other.

### Advantageous Effects of Invention

According to the present invention, it is possible to provide a method for efficiently manufacturing induced pluripotent stem cells.

### BRIEF DESCRIPTION OF DRAWINGS

FIG. 1 shows graphs of the measurement results obtained by a flow cytometer according to Example 1.

FIG. 2 shows graphs of the measurement results obtained by a flow cytometer according to Example 2.

FIG. 3 is an image showing TRA1-60 positive cells according to Example 2.

FIG. 4 shows graphs of the measurement results obtained by a flow cytometer according to Example 3.

FIG. 5 shows graphs of the measurement results obtained by a flow cytometer according to Example 4.

FIG. 6 is a graph showing PCR results according to Example 4.

FIG. 7 shows images of TRA1-60 positive cells according to Example 4.

FIG. 8 shows graphs of the measurement results obtained by a flow cytometer according to Example 5.

FIG. 9 is an image showing cells 15 days after infection according to Example 5.

FIG. 10 shows graphs of the measurement results obtained by a flow cytometer according to Example 5.

FIG. 11 is an image showing cells in the first passage according to Example 5.

FIG. 12 shows graphs of the measurement results obtained by a flow cytometer according to Example 6.

FIG. 13 is an image showing TRA1-60 positive cells according to Example 6.

FIG. 14 shows graphs of the measurement results obtained by a flow cytometer according to Example 7.

FIG. 15 is a graph showing PCR results according to Example 7.

FIG. 16 is an image showing TRA1-60 positive cells according to Example 7.

FIG. 17 shows graphs of the measurement results obtained by a flow cytometer according to Comparative Example 1.

FIG. 18 shows graphs of the measurement results obtained by a flow cytometer according to Comparative Example 2.

FIG. 19 shows graphs of the measurement results obtained by a flow cytometer according to Comparative Example 3.

FIG. 20 shows graphs of the measurement results obtained by a flow cytometer according to Comparative Example 4.

FIG. 21 is a graph showing PCR results according to Example 8.

FIG. 22 shows images of TRA1-60 positive cells according to Example 8.

FIG. 23 is an image of urine-derived cells according to Example 9.

FIG. 24 is an image of urine-derived cells according to Example 9.

FIG. 25 shows images of urine-derived cells transfected with RNA encoding GFP according to Example 10.

FIG. 26 shows images of urine-derived cells into which the reprogramming factor is introduced according to Example 11.

FIG. 27 is an image of urine-derived cells into which the reprogramming factor is introduced according to Example 12.

FIG. 28 shows dot plots obtained by a flow cytometer according to Example 13.

FIG. 29 shows images of urine-derived cells into which the reprogramming factor is introduced according to Example 14.

FIG. 30 shows images of urine-derived cells into which the reprogramming factor is introduced according to Example 14.

### DESCRIPTION OF EMBODIMENTS

Embodiments of the present invention will be described below in detail. Here, the following embodiments exemplify a device and a method for embodying the technical ideas of the invention, and the technical ideas of the invention do not specify the combination of constituent members or the like as in the following. The technical ideas of the invention can be variously modified within the scope of the claims.

A method for manufacturing pluripotent stem cells according to an embodiment includes introducing a reprogramming factor into cells and seeding the cells into which the reprogramming factor is introduced at a low concentration and culturing the cells. Pluripotent stem cells are, for example, iPS cells.

Cells into which the reprogramming factor is introduced are not particularly limited, and examples thereof include fibroblasts, blood cells, dental pulp stem cells, keratinocytes, dermal papilla cells, oral epithelial cells, and somatic prestem cells. Cells into which the reprogramming factor is introduced may be cells contained in urine. Examples of cells contained in urine include bladder epithelial cells. Cells into which the reprogramming factor is introduced may be cells derived from humans or derived from non-human animals. Cells into which the reprogramming factor is introduced may be derived from one human or derived from a plurality of humans. Cells into which the reprogramming factor is introduced may be derived from one non-human animals or derived from a plurality of non-human animals.

Blood cells are isolated from blood. The blood is, for example, peripheral blood or cord blood, but is not limited thereto. Blood may be collected from an adult or a minor. During blood sampling, an anticoagulant such as ethylene-diamine-tetraacetic acid (EDTA), heparin, and a biological preparation standard blood preservative solution A (ACD-A) is used.

Blood cells are nucleated cells, for example, mononuclear cells (monocytes), neutrophilic leukocytes, macrophages, eosinophilic leukocytes, basophil leukocytes, and lymphocytes, and do not include red blood cells, granulocytes, and platelets. Blood cells may be, for example, endothelial progenitor cells, blood stem/progenitor cells, T cells, or B cells. T cells are, for example, αβT cells.

Mononuclear cells are isolated from blood using a medium for isolating blood cells, a centrifugal device or the like. A method for isolating mononuclear cells when Ficoll (GE Healthcare) is used as a medium for isolating blood cells is as follows.

Since the isolation accuracy of mononuclear cells tends to deteriorate at a low temperature, the centrifuge is set at 4° C. to 42° C., preferably 18° C. 10 μL to 50 mL of blood is sampled from an adult or minor human, a chelating agent containing EDTA is added to the blood to prevent the blood from coagulating, and is mixed gently. In addition, 5 mL of a medium for isolating human lymphocytes (Ficoll-Paque PREMIUM, GE Healthcare Japan) is dispensed into two 15 mL tubes. 5 mL of PBS is added to 5 mL of blood for dilution, and 5 mL layers are placed on the medium for isolating human lymphocytes in each of the tubes. In this case, the diluted blood is slowly added onto the medium along the tube wall of the tube to prevent disturbance of the interface.

The solution in the tube is centrifuged at 10×g to 1,000×g, and preferably, 400×g, at 4° C. to 42° C., preferably 18° C., for 5 minutes to 2 hours, preferably for 30 minutes. After centrifugation, a cloudy white intermediate layer appears in the tube. The cloudy white intermediate layer contains mononuclear cells. The cloudy white intermediate layer in the tube is slowly recovered with a Pipeteman and is transferred to a new 15 mL tube. In this case, the lower layer should not be sucked up. About 1 mL of the cloudy white intermediate layer can be recovered from one tube. Two intermediate layers are transferred together into one tube.

1 mL to 48 mL, preferably 12 mL of PBS is added to the recovered mononuclear cells, and the solution is additionally centrifuged at 10×g to 1,000×g, preferably 200×g, at 4° C. to 42° C., preferably 18° C., for 1 minute to 60 minutes, preferably 10 minutes. Then, the supernatant of the solution is sucked up and removed using an aspirator, and 1 mL to 12 mL, preferably 3 mL of a serum-free hematopoietic cell medium of known composition (X-VIVO (registered trademark) 10, Lonza) is added for suspension therein to obtain a mononuclear cell suspension. Of this, 10 μL of a mononuclear cell suspension is stained with trypan blue and counting is performed on a hemacytometer.

A method for isolating mononuclear cells when a Vacutainer (registered trademark, BD) is used as a blood collection tube is as follows.

Since the isolation accuracy of mononuclear cells tends to deteriorate at a low temperature, the centrifuge is set to 4° C. to 42° C., preferably 18° C. 8 mL of blood is sampled from an adult or minor human using a blood collection tube (Vacutainer (registered trademark), BD), mixed by inversion and mixed with an anticoagulant. Then, the balance is adjusted, and the solution is centrifuged at 4° C. to 42° C., preferably 18° C., at 100×g to 3,000×g, preferably 1,500×g to 1,800×g with a swing rotor for 1 minute to 60 minutes, preferably 20 minutes. After centrifugation, the upper layer, which is a plasma layer, is removed and pipetting is performed to suspend the mononuclear cell layer and blood cells adhered to the gel to obtain a suspension. The obtained suspension is transferred to another 15 mL tube.

1 mL to 14 mL, preferably 12 mL of PBS is added to the suspension in a 15 mL tube, and the suspension is centrifuged at 4° C. to 42° C., preferably 18° C., at 100×g to 3,000×g, preferably 200×g for 1 minute to 60 minutes, preferably 5 minutes. After centrifugation, the supernatant is removed with an aspirator. In addition, a hemolytic agent (PharmLyse (registered trademark), 10-fold concentration, BD) is diluted to a 1-fold concentration with sterilized water. The pellet in the 15 mL tube is loosened by tapping, and 1 mL to 14 mL, preferably 1 mL of a hemolytic agent is added. Then, light is blocked therefrom and the solution is left for 1 minute to 60 minutes, preferably 1 minute at room temperature.

Next, 1 mL to 14 mL, preferably 12 mL of PBS is added to a 15 mL tube, and centrifugation is performed at 4° C. to 42° C., preferably room temperature, at 100×g to 3,000×g, preferably 200×g for 1 minute to 60 minutes, or 5 minutes. After centrifugation, the supernatant is removed with an aspirator, and 1 mL to 15 mL, and preferably 3 mL of a serum-free hematopoietic cell medium of known composition (X-VIVO (registered trademark) 10, Lonza) is added for suspension therein to obtain a mononuclear cell suspension. Of this, 10 μL of a mononuclear cell suspension is stained with trypan blue and counting is performed on a hemacytometer.

The method for isolating mononuclear cells from blood is not limited to the above method, and for example, mononuclear cells may be isolated from blood using a dialysis membrane. In addition, Purecell Select System for whole blood mononuclear cell concentration (registered trademark, PALL), a purifier for removing blood cell cells (Cellsorba E, registered trademark, Asahi Kasei), and a filter such as a white blood cell removal filter for platelet preparation (Sepacell PL, registered trademark, PLX-5B-SCD, Asahi Kasei) can also be used.

Mononuclear cells may be isolated using a red blood cell isolating agent that can isolate nucleated cells by gravitational precipitation or centrifugation of red blood cells. Examples of red blood cell isolating agents include HetaSep (registered trademark, STEMCELL Technologies) and HES40 (NIPRO).

In addition, CTL-UP1 (commercially available from Cellular Technology Limited), PBMC-001 (commercially available from Sanguine Biosciences), or the like may be used as mononuclear cells.

Alternatively, regarding the blood cells, blood cells that are cryopreserved using a cell cryopreservation solution such as Cellbanker 1, Stem-Cellbanker GMP grade, or Stem-Cellbanker DMSO free GMP grade (ZENOAQ) may be thawed and used.

When thawing mononuclear cells, first, 1 mL to 15 mL, preferably 8 mL of a serum-free hematopoietic cell medium of known composition (X-VIVO (registered trademark) 10, Lonza) is put into a 15 mL tube, the tube containing frozen mononuclear cells is placed in a warm bath at 4° C. to 42° C., preferably 37° C., and the mononuclear cells start to melt. Then, with the remaining ice, the tube containing mononuclear cells is pulled out of the warm bath, and the mononuclear cells are transferred to a tube containing a serum-free hematopoietic cell medium of known composition. Of this, 10 μL of a mononuclear cell suspension is stained with trypan blue and counting is performed on a hemacytometer.

Blood cells may be isolated based on a cell surface marker. Blood stem/progenitor cells are positive for CD34. T cells are positive for any of CD3, CD4, and CD8. B cells are positive for any of CD10, CD19, and CD20. Macrophages are positive for any of CD11b, CD68, and CD163. Blood stem/progenitor cells, T cells, or B cells are isolated from blood cells using, for example, an automatic magnetic cell isolating device and immunomagnetic beads. Alternatively, mononuclear cells isolated in advance may be prepared. However, blood cells that are not isolated based on a cell surface marker may be used.

CD34 positive cells are stem/progenitor cells, and tend to be easily reprogrammed. In addition, when iPS cells are prepared using T cells which are CD3 positive cells, since the iPS cells derived from T cells maintain a TCR recombination type, differentiation into T cells tends to be efficiently induced.

A method for isolating CD34 positive cells is as follows.

10 μL of IL-6 (100 μg/mL), 10 μL of SCF (300 μg/mL), 10 μL of TPO (300 μg/mL), 10 μL of FLT3 ligands (300 μg/mL), and 10 μL of IL-3 (10 μg/mL) are added to 10 mL of a serum-free medium (StemSpan H3000, STEMCELL Technologies) to prepare a blood cell medium (blood stem/progenitor cell medium).

1 mL to 6 mL, preferably 2 mL of a blood cell medium is put into one well of a 6-well plate. In addition, in order to prevent evaporation of the medium, 1 mL to 6 mL, and 2 mL of PBS are put into the other five wells. Then, the 6-well plate is placed in an incubator at 4° C. to 42° C., preferably 37° C. for warming.

10 μL to 1 mL, preferably 80 μL of EDTA (500 mmol/L) and 10 μL to 1 mL, preferably 200 μL of FBS are added to 20 mL of PBS to prepare a column buffer. A mononuclear cell suspension containing 1×10⁴ to 1×10⁹, preferably 2×10⁷ mononuclear cells is dispensed in a 15 mL tube, and the mononuclear cell suspension is centrifuged at 4° C. to 42° C., preferably 4° C., at 100×g to 3,000×g, preferably 300×g for 10 minutes. After centrifugation, the supernatant is removed, and mononuclear cells are suspended in 100 μL to 1 mL, preferably 300 μL of the column buffer.

10 μL to 1 mL, preferably 100 μL of an FcR blocking reagent (Miltenyi Biotec) and 10 μL to 1 mL, preferably 100 μL of a CD34 microbeads kit (Miltenyi Biotec) are added to the mononuclear cell suspension in the 15 mL tube. The FcR blocking reagent is used to increase the specificity of the microbead labeling. Then, the mononuclear cell suspension is mixed and left at 4° C. to 42° C., preferably 4° C. for 1 minute to 2 hours, preferably 30 minutes.

Next, 1 mL to 15 mL, preferably 10 mL of the column buffer is added to the mononuclear cell suspension in the 15 mL tube and diluted, and centrifugation is performed at 4° C. to 42° C., preferably 4° C., at 100×g to 1,000×g, preferably 300×g for 1 minute to 2 hours, preferably 10 minutes. After centrifugation, the supernatant in the 15 mL tube is removed with an aspirator, and 10 μL to 10 mL, preferably 500 μL of the column buffer is added for resuspension therein.

A column for an automatic magnetic cell isolating device (MS column, Miltenyi Biotec) is attached to an automatic magnetic cell isolating device (MiniMACS Separation Unit, Miltenyi Biotec), and 10 μL to 10 mL, preferably 500 μL of the column buffer is put into the column for washing. Next, mononuclear cells are put into the column. In addition, 10 μL to 10 mL, preferably 500 μL of the column buffer is put into the column, and the column is washed 1 to 10 times, preferably 3 times. Then, the column is removed from the automatic magnetic cell isolating device and put into a 15 mL tube. Next, 10 μL to 10 mL, preferably 1,000 μL of the column buffer is put into the column, and the syringe is immediately pushed to discharge CD34 positive cells to the 15 mL tube.

10 μL of a CD34 positive cell suspension is stained with trypan blue and the number of cells is counted on a blood cell counting chamber. In addition, the CD34 positive cell suspension in the 15 mL tube is centrifuged at 4° C. to 42° C., preferably 4° C., at 100×g to 1,000×g, preferably 300×g for 1 minute to 2 hours, preferably 10 minutes. After centrifugation, the supernatant is removed with an aspirator. In addition, CD34 positive cells are re-suspended in the warmed blood cell medium and the CD34 positive cells are sprinkled on a culture plate. Then, the CD34 positive cells are cultured for 6 days at 4° C. to 42° C., preferably 37° C., in 1% to 203, preferably 5% CO₂. During this time, the medium does not have to be replaced.

A method for isolating cells with a marker other than CD34 is the same as a method for isolating CD34 positive cells.

The reprogramming factor introduced into cells is, for example, RNA. The RNA is, for example, mRNA. Examples of reprogramming factors introduced into cells include OCT RNA such as OCT3/4, SOX RNA such as SOX2, KLF RNA such as KLF4, and MYC RNA such as c-MYC. As reprogramming factor RNA, M₃O which is improved OCT3/4 may be used. In addition, the reprogramming factor RNA may further include RNA of at least one factor selected from the group consisting of LIN28A, FOXH1, LIN28B, GLIS1, p53-dominant negative, p53-P275S, L-MYC, NANOG, DPPA2, DPPA4, DPPA5, ZIC3, BCL-2, E-RAS, TPT1, SALL2, NAC1, DAX1, TERT, ZNF206, FOXD3, REX1, UTTF1, KLF2, KLF5, ESRRB, miR-291-3p, miR-294, miR-295, NR5A1, NR5A2, TBX3, MBD3sh, TH2A, TH2B, and P53DD. These RNAs are available from TriLink. Here, although the gene symbols are denoted here as those of humans, this is not intended to limit the species by uppercase or lowercase letters. For example, denoting in all uppercase letters does not exclude inclusion of mouse or rat genes. However, in the examples, the gene symbols are shown according to the species actually used.

RNA may be modified with pseudouridine (ψ) or 5-methyluridine (5meU). RNA may be polyadenylated.

The reprogramming factor is introduced into cells, for example, by a lipofection method. The lipofection method is a method in which a complex of a nucleic acid, which is a negatively charged substance, and a positively charged lipid, is formed by an electrical interaction, and the complex is incorporated into cells by endocytosis or membrane fusion. The lipofection method has advantages such as less damage to cells, excellent introduction efficiency, ease of operation, and less time-consumption.

For example, the reprogramming factor is introduced into cells cultured using an RNA transfection reagent. For example, when cells are mononuclear cells, immediately after mononuclear cells are isolated from blood, RNA may be introduced into the mononuclear cells.

Lipofectamine MessengerMAX (registered trademark, Thermo Fisher SCIENTIFIC) can be used as the RNA transfection reagent. Alternatively, regarding the RNA transfection reagent, for example, a lipofection reagent such as Lipofectamine (registered trademark) RNAiMAX (Thermo Fisher SCIENTIFIC), Lipofectamine StemTransfection Reagent (Thermo Fisher SCIENTIFIC), TransIT (Mirus), mRNA-In (MTI-GlobalStem), Stemfect RNA Transfection Kit (ReproCELL), Jet Messenger (Polyplus), Lipofectamin (registered trademark) 2000, Lipofectamin (registered trademark) 3000, NeonTransfection System (Thermo Fisher SCIENTIFIC), Stemfect RNA transfection reagent (Stemfect), NextFect (registered trademark) RNA Transfection Reagent (BiooSientific), Amaxa (registered product) Human T cell Nucleofector (registered product) kit (Lonza, VAPA-1002), Amaxa (registered product) Human CD34 cell Nucleofector (registered product) kit (Lonza, VAPA-1003), and ReproRNA (registered trademark) transfection reagent (STEMCELL Technologies) may be used.

Alternatively, for example, a reprogramming factor is introduced into cells using a viral vector. The viral vector may be an RNA viral vector. The RNA viral vector may be a Sendai viral vector. The Sendai viral vector may be a temperature-sensitive Sendai viral vector in which the stability of a viral nucleic acid decreases at a predetermined temperature or higher. The viral nucleic acid of the temperature-sensitive Sendai viral vector is stable below a predetermined temperature. The viral nucleic acid may be viral DNA or viral RNA. The viral nucleic acid may be a virus genome. The decrease in the stability of the viral nucleic acid may be at least one of decomposition of the viral nucleic acid and minimization of replication or proliferation of the viral nucleic acid. When the stability of the viral nucleic acid decreases, at least one of proliferation of the viral nucleic acid, the replication rate of the viral nucleic acid and the gene expression level decreases. The predetermined temperature is, for example, 36.5° C. or higher and 37.5° C. or lower, 36.6° C. or higher and 37.4° C. or lower, 36.7° C. or higher and 37.3° C. or lower, 36.8° C. or higher and 37.2° C. or lower, 36.9° C. or higher and 37.1° C. or lower, or 37° C. The stability of the viral nucleic acid of the temperature-sensitive Sendai viral vector, that is, at least one of the proliferation, the replication rate and the gene expression level, is high at a temperature lower than a predetermined temperature, and low at a predetermined temperature or higher. For example, in the temperature-sensitive Sendai viral vector, the proliferation rate or the gene expression level in cells cultured at 37° C. is ½ or less, ⅓ or less, ⅕ or less, 1/10 or less, or 1/20 or less with respect to the proliferation rate or the gene expression level in cells cultured at 32° C.

The Sendai virus encodes the N gene, P gene, M gene, F/HN gene, and L gene. The HN protein recognizes sialic acid on the cell surface when the Sendai virus attaches to cells and fixes virus particles to the cells. The F protein is cleaved and activated with extracellular proteases, and catalyzes the fusion of the fixed Sendai virus envelope and the cell membrane of target cells to establish infection. Along with its modified protein, that is, the P protein, the L protein catalyzes replication of viral nucleic acids in the cytoplasm after infection and transcription from the replicated multi-copy nucleic acids.

When the F gene is deleted in the Sendai viral vector, it is possible to restrict production of infectious virus particles from transgenic cells. In addition, when a mutation is introduced into at least one of the L gene and P gene, it is possible to make the Sendai viral vector temperature sensitive.

Examples of temperature-sensitive (TS) mutation of the Sendai virus include TS7 (Y942H/L1361C/L1558I mutation of the L protein), TS12 (D433A/R434A/K437A mutation of the P protein), TS13 (D433A/R434A/K437A mutation of the P protein and L1558I mutation of the L protein), TS14 (D433A/R434A/K437A mutation of the P protein and L1361C mutation of the L protein), and TS15 (D433A/R434A/K437A mutation of the P protein and L1361C/L1558I mutation of the L protein).

The Sendai viral vector is, for example, an F gene-deficient (ΔF) Sendai viral vector having G69E, T116A, and A183S mutations in the M protein, A262T, G264R, and K461G mutations in the HN protein, L511F mutation in the P protein, and N1197S and K1795E mutations in the L protein, which is a Sendai viral vector into which the TS7, TS12, TS13, TS14, or TS15 mutation is introduced. However, the temperature-sensitive mutation of the Sendai viral vector is not limited thereto.

The Sendai viral vector is, for example, SeV(PM)/TSΔF, SeV18+/TSΔF, or SeV(HNL)/TSΔF, and is a Sendai viral vector into which the TS7, TS12, TS13, TS14, or TS15 mutation is introduced. However, the temperature-sensitive mutation of the Sendai viral vector is not limited thereto.

The Sendai viral vector introduced into cells may be a combination of a temperature-sensitive Sendai viral vector and a temperature-insensitive Sendai viral vector. Alternatively, the Sendai viral vector introduced into cells may be a temperature-sensitive Sendai viral vector only and may not include a temperature-insensitive Sendai viral vector. For example, the Sendai viral vector introduced into cells may be only a temperature-sensitive Sendai viral vector into which the TS7, TS12, TS13, TS14, or TS15 mutation is introduced and may not include a temperature-insensitive Sendai viral vector. For example, the Sendai viral vector introduced into cells may be only a Sendai viral vector having a temperature sensitivity equal to or higher than that of a temperature-sensitive Sendai viral vector into which the TS7, TS12, TS13, TS14, or TS15 mutation is introduced, and may not include a temperature-insensitive Sendai viral vector. For example, the Sendai viral vector introduced into cells may be only a Sendai viral vector having a temperature sensitivity equal to or higher than that of a temperature-sensitive Sendai viral vector into which the TS7, TS12, TS13, TS14, or TS15 mutation is introduced, and may not include a Sendai viral vector having a lower temperature sensitivity than a temperature-sensitive Sendai viral vector into which the TS7, TS12, TS13, TS14, or TS15 mutation is introduced.

The Sendai viral vector introduced into cells carries arbitrary reprogramming factors. The Sendai viral vector introduced into cells may be, for example, a combination of a temperature-sensitive Sendai viral vector including KLF RNA, OCT RNA, and SOX RNA in that order and not including MYC RNA, and a temperature-sensitive Sendai viral vector including MYC RNA and not including KLF RNA, OCT RNA, and SOX RNA. However, the number, combination, and order of reprogramming factors carried on the Sendai viral vector are arbitrary, and are not particularly limited.

The Sendai viral vector introduced into cells may include a Sendai viral vector including KLF RNA and not including OCT RNA and SOX RNA. The Sendai viral vector including KLF RNA and not including OCT RNA and SOX RNA may be a temperature-sensitive Sendai viral vector or a temperature-insensitive Sendai viral vector. However, according to the findings of the inventors, if a temperature-insensitive Sendai viral vector is not introduced, the Sendai viral vector disappears earlier from the cells into which the Sendai viral vector is introduced.

The temperature-sensitive Sendai viral vector including KLF RNA, OCT RNA, and SOX RNA is, for example, an F gene-deficient Sendai viral vector having G69E, T116A, and A183S mutations in the M protein, A262T, G264R, and K461G mutations in the HN protein, L511F mutation in the P protein, and N1197S and K1795E mutations in the L protein, which is a Sendai viral vector including the TS7, TS12, TS13, TS14, or TS15 mutation. The temperature-sensitive mutation is, for example, TS7 or TS12, or TS12.

The temperature-sensitive Sendai viral vector including KLF RNA, OCT RNA, and SOX RNA is, for example, SeV(PM)KOS/TS7ΔF or SeV(PM)KOS/TS12ΔF, or SeV(PM)KOS/TS12ΔF.

The temperature-sensitive Sendai viral vector including MYC RNA is, for example, an F gene-deficient Sendai virus vector having G69E, T116A, and A183S mutations in the M protein, A262T, G264R, and K461G mutations in the HN protein, L511F mutation in the P protein, and N1197S and K1795E mutations in the L protein, which is a Sendai viral vector including the TS7, TS12, TS13, TS14, or TS15 mutation. The temperature-sensitive mutation is, for example, TS15.

The temperature-sensitive Sendai viral vector including MYC RNA is, for example, SeV(HNL)MYC/TS12ΔF, SeV(HNL)MYC/TS13ΔF, or SeV(HNL)MYC/TS15ΔF, or SeV(HNL)MYC/TS15ΔF.

The Sendai viral vector including KLF RNA and not including OCT RNA and SOX RNA is, for example, an F gene-deficient Sendai viral vector having G69E, T116A, and A183S mutations in the M protein, A262T, G264R, and K461G mutations in the HN protein, L511F mutation in the P protein, and N1197S and K1795E mutations in the L protein. The Sendai viral vector including KLF RNA and not including OCT RNA and SOX RNA is less temperature-sensitive than, for example, a Sendai viral vector into which the TS7, TS12, TS13, TS14, or TS15 mutation is introduced and can express the KLF gene at a predetermined temperature or higher.

The Sendai viral vector including KLF RNA and not including OCT RNA and SOX RNA is, for example, SeV18+KLF4/TSΔF.

When a plurality of types of Sendai viral vectors are introduced into cells, for example, a plurality of types of Sendai viral vectors are introduced into cells at the same time. Alternatively, within 48 hours, within 36 hours, within 24 hours, within 18 hours, within 12 hours, within 10 hours, within 8 hours, within 6 hours, within 3 hours, within 2 hours, or within 1 hour after a certain type of Sendai viral vector is introduced into cells, it is preferable to introduce all types of Sendai viral vectors into cells.

The multiplicity of infection (MOI) of the Sendai viral vector when cells are infected is, for example, 0.1 or more, 0.3 or more, 0.5 or more, 1.0 or more, 2.0 or more, 3.0 or more, 4.0 or more, or 5.0 or more. In addition, the MOI is, for example, 100 or less, 90 or less, 80 or less, 70 or less, 60 or less, 50 or less, 40 or less, 30 or less, 20 or less, 10 or less, or 5 or less.

The temperature at which cells are infected with a Sendai viral vector may be lower than a predetermined temperature at which the stability of the viral nucleic acid of the temperature-sensitive Sendai viral vector decreases, that is, a temperature at which the viral nucleic acid of the temperature-sensitive Sendai viral vector is stable, or a predetermined temperature or higher. When the Sendai viral vector is only a temperature-sensitive Sendai viral vector and does not include a temperature-insensitive Sendai viral vector, the temperature at which cells are infected with a Sendai viral vector is preferably a temperature that is lower than a predetermined temperature at which the stability of the viral nucleic acid of the temperature-sensitive Sendai viral vector decreases, that is, that is, a temperature at which the viral nucleic acid of the temperature-sensitive Sendai viral vector is stable.

Cells into which the reprogramming factor is introduced may be adherently cultured or suspension-cultured.

Somatic cells into which the reprogramming factor is introduced may be feeder-free cultured using a basement membrane matrix such as Matrigel (Corning), CELLstart (registered trademark, ThermoFisher), Laminin 511 (iMatrix-511, nippi), fibronectin, and vitrotin.

As the medium in which cells into which the reprogramming factor is introduced are cultured, a stem cell medium such as a human ES/iPS medium, for example, Primate ES Cell Medium (ReproCELL), can be used.

However, the stem cell medium is not limited thereto and various stem cell mediums can be used. For example, Primate ES Cell Medium, Reprostem, ReproFF, ReproFF2, ReproXF (Reprocell), mTeSR1, TeSR2, TeSRE8, ReproTeSR (STEMCELL Technologies), PluriSTEM (registered trademark) Human ES/iPS Medium (Merck), NutriStem (registered trademark) XF/FF Culture Medium for Human iPS and ES Cells, Pluriton reprogramming medium (Stemgent), PluriSTEM (registered trademark), Stemfit AK02N, Stemfit AK03 (Ajinomoto), ESC-Sure (registered trademark) serum and feeder free medium for hESC/iPS (Applied StemCell), L7 (registered trademark) hPSC Culture System (LONZA), and PluriQ (MTI-GlobalStem) may be used. The stem cell medium preferably contains albumin with a reduced amount of fatty acids supported. The stem cell medium is put into an incubator, for example, a dish, a well, or a tube.

When cells are suspension-cultured or three-dimensionally cultured, for example, a gel medium is used. For example, the gel medium is prepared by adding gellan gum to a stem cell medium so that the final concentration is 0.001 mass, to 0.5 mass %, 0.005 mass % to 0.1 mass %, or 0.01 mass % to 0.05 mass %.

The gel medium may contain at least one polymer compound selected from the group consisting of gellan gum, hyaluronic acid, ramsan gum, diutan gum, xanthan gum, carrageenan, fucoidan, pectin, pectic acid, pectinic acid, heparan sulfate, heparin, heparitin sulfate, keratosulfate, chondroitin sulfate, dermatan sulfate, rhamnan sulfate, and salts thereof. In addition, the gel medium may contain methyl cellulose. When the gel medium contains methyl cellulose, aggregation between cells is further reduced.

Alternatively, the gel medium may contain a small amount of a temperature-sensitive gel selected from among poly(glycerol monomethacrylate) (PGMA), poly(2-hydroxypropyl methacrylate) (PHPMA), Poly(N-isopropylacrylamide) (PNIPAM), amine terminated, carboxylic acid terminated, maleimide terminated, N-hydroxysuccinimide (NHS) ester terminated, triethoxysilane terminated, Poly(N-isopropylacrylamide-co-acrylamide), Poly(N-isopropylacrylamide-co-acrylic acid), Poly(N-isopropylacrylamide-co-butylacrylate), Poly(N-isopropylacrylamide-co-methacrylic acid), Poly(N-isopropylacrylamide-co-methacrylic acid-co-octadecyl acrylate), and N-Isopropylacrylamide.

The gel medium may or may not contain a growth factor, for example, a basic fibroblast growth factor (bFGF). Alternatively, the gel medium may contain a growth factor such as bFGF at a low concentration of 400 μg/L or less, 40 μg/L or less, or 10 μg/L or less.

In addition, the gel medium may or may not contain TGF-β, and may contain TGF-β at a low concentration of 600 μg/L or less, 300 μg/L or less, or 100 μg/L or less.

The gel medium may not be stirred. In addition, the gel medium may not contain feeder cells.

The gel medium may contain at least one substance selected from the group consisting of cadherin, laminin, fibronectin, and vitronectin.

After cells are infected with a Sendai viral vector, for at least 2 days, or 2 days or more and 10 days or less, the cells may be cultured at a temperature lower than a predetermined temperature at which the stability of the viral nucleic acid of the temperature-sensitive Sendai viral vector decreases, that is, a temperature at which the viral nucleic acid of the temperature-sensitive Sendai viral vector is stable. Then, the cells may be cultured at a predetermined temperature or higher. When the cells are cultured at a predetermined temperature or higher, for example, the medium may be replaced once every two days.

After cells are infected with a Sendai viral vector, the cells may be cultured for at least 2 days, or 2 days or more and 10 days or less, for example, at a temperature of 4.0° C. or higher, 10° C. or higher, 15° C. or higher, 20° C. or higher, 25° C. or higher, 30° C. or higher, 31.0° C. or higher, 32.0° C. or higher, 33.0° C. or higher, 33.1° C. or higher, 33.2° C. or higher, 33.3° C. or higher, 33.4° C. or higher, 33.5° C. or higher, 33.6° C. or higher, 33.7° C. or higher, 33.8° C. or higher, or 33.9° C. or higher, lower than 37.0° C., lower than 36.9° C., lower than 36.8° C., lower than 36.7° C., lower than 36.6° C., lower than 36.5° C., 36.0° C. or lower, 35.0° C. or lower, or 34.0° C. or lower. Then, the temperature is raised, and the cells may be cultured at a temperature of 36.5° C. or higher, 36.6° C. or higher, 36.7° C. or higher, 36.8° C. or higher, 36.9° C. or higher, or 37.0° C. or higher, and 40.0° C. or lower, 39.0° C. or lower, or 38.0° C. or lower. The temperature may be raised once or stepwise. After the temperature is raised, when the cells are cultured, for example, the medium may be replaced once every two days.

After cells are infected with a Sendai viral vector, until stem-cell-like colonies begin to appear, the cells may be cultured at a temperature lower than a predetermined temperature at which the stability of the viral nucleic acid of the temperature-sensitive Sendai viral vector decreases, that is, a temperature at which the viral nucleic acid of the temperature-sensitive Sendai viral vector is stable. After the stem-cell-like colonies begin to appear, the cells may be cultured at a predetermined temperature or higher. When the cells are cultured at a predetermined temperature or higher, for example, the medium may be replaced once every two days.

After cells are infected with a Sendai viral vector, until stem-cell-like colonies begin to appear, the cells may be cultured at a temperature of, for example, 4.0° C. or higher, 10° C. or higher, 15° C. or higher, 20° C. or higher, 25° C. or higher, 30° C. or higher, 31.0° C. or higher, 32.0° C. or higher, 33.0° C. or higher, 33.1° C. or higher, 33.2° C. or higher, 33.3° C. or higher, 33.4° C. or higher, 33.5° C. or higher, 33.6° C. or higher, 33.7° C. or higher, 33.8° C. or higher, or 33.9° C. or higher, and lower than 37.0° C., lower than 36.9° C., lower than 36.8° C., lower than 36.7° C., lower than 36.6° C., lower than 36.5° C., 36.0° C. or lower, 35.0° C. or lower, or 34.0° C. or lower. After the stem-cell-like colonies begin to appear, the temperature is raised, and the cells may be cultured at a temperature of 36.5° C. or higher, 36.6° C. or higher, 36.7° C. or higher, 36.8° C. or higher, 36.9° C. or higher, or 37.0° C. or higher, and 40.0° C. or lower, 39.0° C. or lower, or 38.0° C. or lower. The temperature may be raised once or stepwise. After the temperature is raised, when the cells are cultured, for example, the medium may be replaced once every two days.

After cells are infected with a Sendai viral vector and the cells are cultured, the cells are passaged at least once. The passage may be performed a plurality of times. When cells into which the reprogramming factor is introduced are passaged using a Sendai viral vector, the cells are seeded in a medium or an incubator at a low concentration. Here, the low concentration is, for example, 1 cell/cm² or more, 0.25×10⁴ cells/cm² or less, 1.25×10³ cells/cm² or less, 0.25×10³ cells/cm² or less, 0.25×10² cells/cm² or less, or 0.25×10¹ cells/cm² or less. Alternatively, the low concentration is a concentration at which 10 cells or less, 9 cells or less, 8 cells or less, 7 cells or less, 6 cells or less, 5 cells or less, 4 cells or less, 3 cells or less, or 2 cells or less can come into contact with each other and 11 cells or more do not come into contact with each other. Here, there may be a plurality of cell masses in which 10 cells or less come into contact with each other. Alternatively, the state in which the entire bottom surface of the cell container is covered with cells is regarded as 100% confluency, and the low concentration is 5% or less confluency, 4% or less confluency, 3% or less confluency, 25 or less confluency, 1% or less confluency, 0.5% or less confluency, 0.1% or less confluency, 0.05% or less confluency, or 0.01% or less confluency. In addition, alternatively, the low concentration is, for example, a concentration at which single cells do not come into contact with each other in the seeded cells. For example, single cells may be seeded in wells of a well plate. The well plate may be a 12-well plate or a 96-well plate. According to the findings of the inventors, when cells into which the reprogramming factor is introduced are passaged, cells are seeded in a medium at a low concentration, and thus the residual Sendai virus in pluripotent stem cells induced from cells can be minimized. The proportion of cells in which the Sendai virus remains among the induced pluripotent stem cells is, for example, 4% or less, 3% or less, 2% or less, 1% or less, 0.5% or less, or 0%.

After passage, cells may be cultured at a predetermined temperature or higher at which the stability of the viral nucleic acid of the temperature-sensitive Sendai viral vector decreases. After passage, for example, cells may be cultured at a temperature of 36.5° C. or higher and lower than 38.0° C. After passage, for example, cells are cultured at a temperature of 36.5° C. or higher and lower than 38.0° C. until intercellular adhesion starts, and after intercellular adhesion starts, until intercellular adhesion starts, cells may be cultured at a higher temperature, for example, at a temperature of 37.5° C. or higher, 42.0° C. or lower, 41.5° C. or lower, 41.0° C. or lower, 40.5° C. or lower, or 40.0° C. or lower. After passage, before intercellular adhesion starts, cells may be cultured at a temperature of 37.5° C. or higher, 42.0° C. or lower, 41.5° C. or lower, 41.0° C. or lower, 40.5° C. or lower, or 40.0° C. or lower.

In the prior art, when a reprogramming factor is introduced into cells to induce the cells to pluripotent stem cells, since many cells that have not been induced to stem cells remain, it is necessary to isolate stem-cell-like colonies from cells that are not induced to stem cells. Specifically, it is necessary to pick up and passage stem-cell-like colonies with a pipette while observed them under a microscope or the like. On the other hand, according to the method for the present embodiment, cells into which a reprogramming factor has been introduced are highly efficiently induced to pluripotent stem cells. Therefore, cells that have not been induced to stem cells do not substantially remain or remain in a few percent or less so that stem-cell-like colonies do not have to be isolated from cells that have not been induced to stem cells during passage and cryopreservation.

According to the method for the present embodiment, since cells that have not been induced to stem cells do not substantially remain or remain in a few percent or less, during passage, cells into which the reprogramming factor is introduced may be recovered, and at least some of the recovered and mixed cells may be seeded in a medium. In the passage, clones of cells into which the reprogramming factor is introduced may be mixed. In the passage, different clones of cells into which the reprogramming factor is introduced may be mixed. Then, cells into which the reprogramming factor is introduced are recovered and at least some of the recovered and mixed cells are seeded and passaged in a medium, which may be performed a plurality of times. Until stem cells are derived, cells into which the reprogramming factor is introduced may be recovered, and at least some of the recovered and mixed cells may be seeded and passaged in a medium. Here, all the recovered and mixed cells may be seeded in a medium.

Here, recovering cells into which the reprogramming factor is introduced and seeding and passaging at least some of the recovered and mixed cells in a medium refers to, for example, passaging cells into which the reprogramming factor is introduced without distinguishing them according to the gene expression state. For example, during passage, cells into which the reprogramming factor is introduced may be seeded in the same incubator without distinguishing them according to the gene expression state. Alternatively, recovering cells into which the reprogramming factor is introduced and seeding and passaging at least some of the recovered and mixed cells in a medium refers to, for example, passaging cells into which the reprogramming factor is introduced without distinguishing them according to the degree of reprogramming. For example, during passage, cells into which the reprogramming factor is introduced may be seeded in the same incubator without distinguishing them according to the degree of reprogramming.

Alternatively, recovering cells into which the reprogramming factor is introduced and seeding and passaging at least some of the recovered and mixed cells in a medium refers to, for example, passaging cells into which the reprogramming factor is introduced without distinguishing them according to the form. For example, during passage, cells into which the reprogramming factor is introduced may be seeded in the same incubator without distinguishing them according to the form. Alternatively, recovering cells into which the reprogramming factor is introduced and seeding and passaging at least some of the recovered and mixed cells in a medium refers to, for example, passaging cells into which the reprogramming factor is introduced without distinguishing them according to the size. For example, during passage, cells into which the reprogramming factor is introduced may be seeded in the same incubator without distinguishing them according to the size.

In addition, alternatively, recovering cells into which the reprogramming factor is introduced and seeding and passaging at least some of the recovered and mixed cells in a medium refers to passaging without cloning cells into which the reprogramming factor is introduced. For example, when passaging is performed without cloning, it is not necessary to pick up colonies formed by cells into which the reprogramming factor is introduced. For example, when passaging is performed without cloning, a plurality of colonies formed by cells into which the reprogramming factor is introduced may not be isolated from each other. For example, during passage, cells forming a plurality of different colonies may be mixed and seeded in the same incubator. In addition, for example, when passaging is performed without cloning, it is not necessary to clone a single colony formed by cells into which the reprogramming factor is introduced. For example, during passage, colonies may be mixed and seeded in the same incubator.

For example, when cells into which the reprogramming factor is introduced are adherently cultured, cells that are adherently cultured may be recover, and at least some of the recovered and mixed cells may be seeded and passaged in a medium. For example, during passage, cells may be separated from the incubator, and at least some of the separated and mixed cells may be seeded in the same incubator. For example, cells may be separated from the incubator with a separation solution and all separated and mixed cells may be passaged. For example, cells that do not form colonies may be passaged. When cells into which the reprogramming factor is introduced are suspension-cultured, all suspension-cultured cells may be passaged.

For example, when a reprogramming factor is introduced but cells are adherently cultured, all adherently cultured cells may be passaged. For example, all cell separated from the incubator with a separation solution may be passaged. In addition, after cells into which the reprogramming factor is introduced are induced to pluripotent stem cells, all adherently cultured cells may be cryopreserved as pluripotent stem cells. For example, all cells separated from the incubator with a separation solution may be cryopreserved as pluripotent stem cells.

When cells into which the reprogramming factor is introduced are suspension-cultured, all suspension-cultured cells may be passaged. In addition, after cells into which the reprogramming factor is introduced are induced to pluripotent stem cells, all suspension-cultured cells may be cryopreserved as pluripotent stem cells.

In the pluripotent stem cells induced by the method according to the present embodiment, since the Sendai virus disappears equally, a plurality of formed colonies may be mixed with each other. In any of the plurality of colonies of pluripotent stem cells induced by the method according to the present embodiment, since cells that have not been induced to stem cells do not substantially remain or remain in a few percent or less, the plurality of formed colonies may be mixed with each other.

The induced pluripotent stem cells can form flat colonies similar to ES cells and express alkaline phosphatase. The induced pluripotent stem cells can express undifferentiated cell markers Nanog, OCT4, SOX2 and the like. The induced pluripotent stem cells can express TERT. The induced pluripotent stem cells can exhibit telomerase activity.

In addition, determination of whether cells are induced to pluripotent stem cells may be performed by analyzing whether at least one surface marker selected from among cell surface markers TRA-1-60, TRA-1-81, SSEA-1, and SSEA5 which indicate undifferentiation, with a cyto flowmeter, is positive. TRA-1-60 is an antigen specific for iPS/ES cells. Since iPS cells can be produced only from TRA-1-60 positive fractions, TRA-1-60 positive cells are considered to be the species of iPS cells.

### EXAMPLES

### Example 1

A dish coated with laminin 511 was used as a dish for inducing pluripotent stem cells. In addition, human peripheral blood mononuclear cells were suspended in a blood medium, the number of mononuclear cells was measured using a blood cell counting chamber, and the number of mononuclear cells in the blood medium was adjusted. Then, the mononuclear cells were two-dimensionally cultured on the dish for inducing pluripotent stem cells at 37° C. for 1 to 7 days.

CytoTune-iPS2.0 (registered trademark, ID Pharma), which is a Sendai viral vector kit, was prepared. CytoTune-iPS2.0 includes SeV(PM)hKOS/TS12ΔF, which is a temperature-sensitive Sendai viral vector that carries KLF4 genes, OCT3/4 genes, and SOX2 genes as reprogramming factors, SeV18+hKLF4/TSΔF, which is a temperature-sensitive Sendai viral vector that carries KLF4 as a reprogramming factor, and SeV(HNL)hC-Myc/TS15ΔF, which is a temperature-sensitive Sendai viral vector that carries c-MYC as a reprogramming factor.

SeV(PM)hKOS/TS12ΔF, SeV18+hKLF4/TSΔF, and SeV(HNL)hC-Myc/TS15ΔF were added to mononuclear cells so that the MOI was 5, a dish for inducing pluripotent stem cells was accommodated in an incubator at 37° C. and the cells were cultured. Two days after infection, the blood medium was replaced with an iPS cell medium. Then, the medium was replaced once every two days using the iPS cell medium.

8 days after infection, stem-cell-like cell masses generated. On the 14th day after infection, almost all cells became TRA1-60 positive cells, and showed iPS-cell-like morphology. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, a ROCK inhibitor was additionally added, and a cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Then, the well-dish was accommodated in an incubator at 37° C., and cells were two-dimensionally cultured. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other.

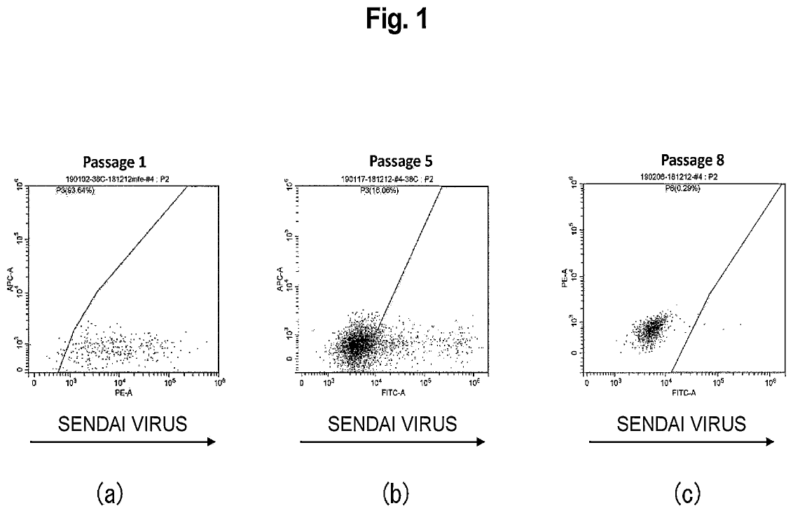

As shown in FIG. 1, when the cells were stained using anti-Sendai virus antibodies and the Sendai virus remaining in the cells was evaluated with a flow cytometer, at the 8th passage, the Sendai virus in the cells almost disappeared.

### Example 2

SeV(PM)hKOS/TS12ΔF, SeV18+hKLF4/TSΔF, and SeV(HNL)hC-Myc/TS15ΔF were added to two-dimensionally cultured fibroblasts so that the MOI was 5, a dish for inducing pluripotent stem cells was accommodated in an incubator at 34° C., and the cells were cultured. Two days after infection, the blood medium was replaced with an iPS cell medium. Then, the medium was replaced once every two days using the iPS cell medium. On the 14th day after infection, the culture temperature was gradually raised to 37° C. and 38° C.

10 days after infection, stem-cell-like cell masses generated. On the 14th day after infection, almost all cells became TRA1-60 positive cells, and showed iPS-cell-like morphology. 14 days after infection, a TrypLE select as a cell-releasing agent was added to the dish, and a cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Next, the well-dish was accommodated in an incubator, and the cells were two-dimensionally cultured. After the cells began to divide, the culture temperature was set to 38° C. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other.

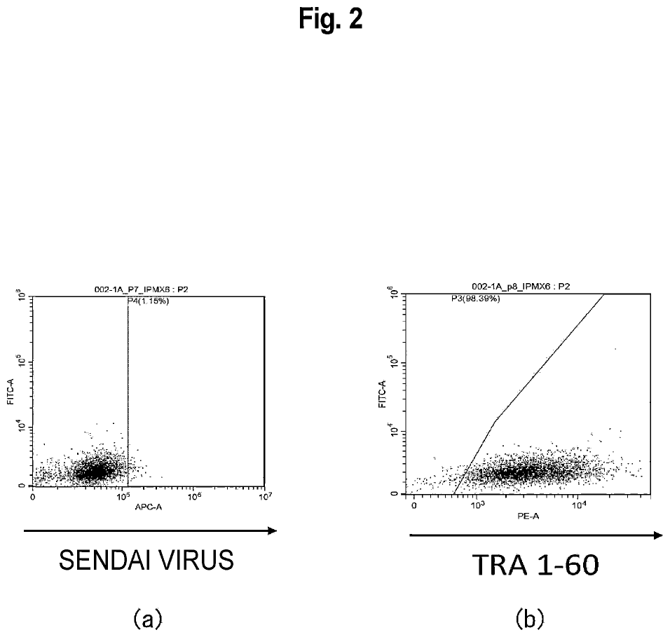

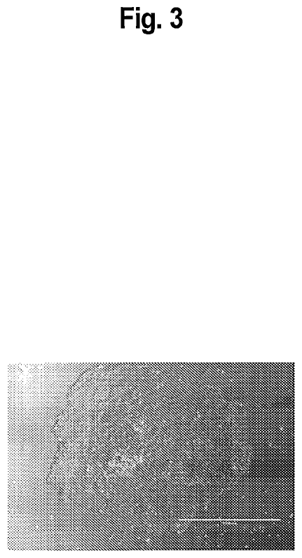

As shown in FIG. 2, when the cells that had been passaged only once were stained with anti-Sendai virus antibodies and the Sendai virus remaining in the cells was evaluated with a flow cytometer, the Sendai virus in the cells was almost disappeared. FIG. 3 shows an image of the obtained TRA1-60 positive cells.

### Example 3

SeV(PM)hKOS/TS12ΔF, SeV18+hKLF4/TSΔF, and SeV(HNL)hC-Myc/TS15ΔF were added to two-dimensionally cultured mononuclear cells so that the MOI was 5, a dish for inducing pluripotent stem cells was accommodated in an incubator at 37° C. and the cells were cultured. Two days after infection, the blood medium was replaced with an iPS cell medium. Then, the medium was replaced once every two days using the iPS cell medium.

8 days after infection, stem-cell-like cell masses generated. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The concentration of the cell-containing solution was adjusted so that 11 or more cells adhered to each other, and the cells were seeded in a well plate for the first passage so that the concentration was higher than 0.25×10⁴ cells/cm². Next, the well-dish was accommodated in an incubator at 37° C., and cells were two-dimensionally cultured. After the cells began to divide, the culture temperature was raised to 38° C. Then, the cells were passaged so that the cells had 60% to 80% confluency. From the second passage to the fifth passage, 11 or more cells adhered to each other, and the cells were seeded in a well plate so that the concentration was higher than 0.25×10⁴ cells/cm². From the sixth passage onward, the cells were seeded in a well plate so that the concentration was 0.25-10⁴ cells/cm² or less. In this case, 11 or more cells did not come into contact with each other.

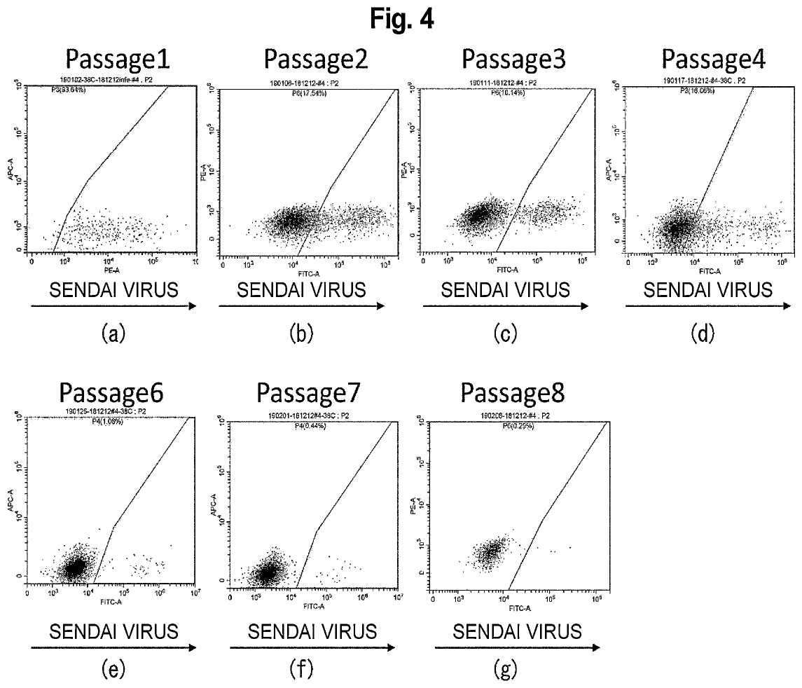

As shown in FIG. 4, when the cells were stained using anti-Sendai virus antibodies and the Sendai virus remaining in the cells was evaluated with a flow cytometer, the Sendai virus remained in the cells before the sixth passage. However, in the sixth passage onward in which the cells were seeded in a well plate so that the concentration was 0.25×10⁴ cells/cm² or less, the Sendai virus in the cells rapidly disappeared.

### Example 4

SeV(PM)hKOS/TS12ΔF and SeV(HNL)hC-Myc/TS15ΔF were added to two-dimensionally cultured mononuclear cells so that the MOI was 5, the dish for inducing pluripotent stem cells was accommodated in an incubator at 34° C., and the cells were cultured. Two days after infection, the blood medium was replaced with an iPS cell medium. Then, the medium was replaced once every two days using the iPS cell medium. Along the way, the temperature was gradually raised to 37° C., 38° C.

8 days after infection, stem-cell-like cell masses generated. On the 14th day after infection, almost all cells became TRA1-60 positive cells, and showed iPS-cell-like morphology. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Next, the well-dish was accommodated in an incubator at 37° C., and cells were two-dimensionally cultured. After the cells began to divide, the culture temperature was raised to 38° C. Then, the cells were passaged so that the cells had 60C to 80% confluency. From the second passage onward, the cells were seeded in the well plate so that the concentration was 0.25×10⁴ cells/cm² or less. In this case also, 11 or more cells did not come into contact with each other.

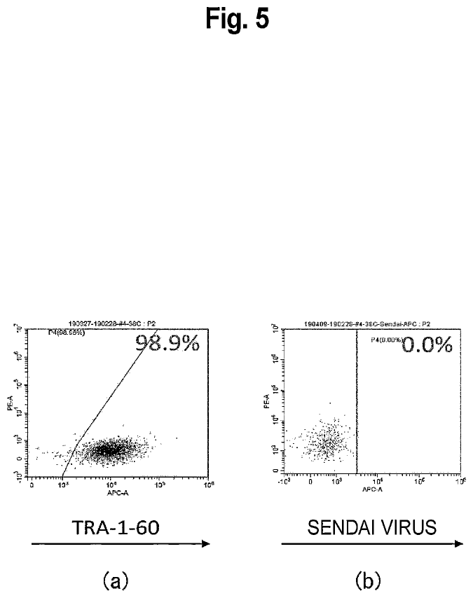

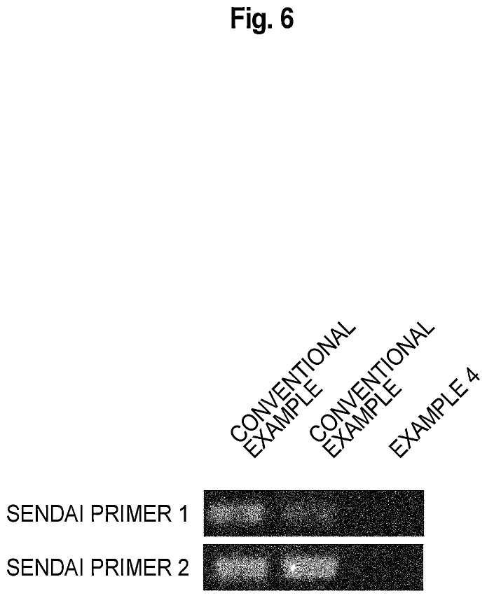

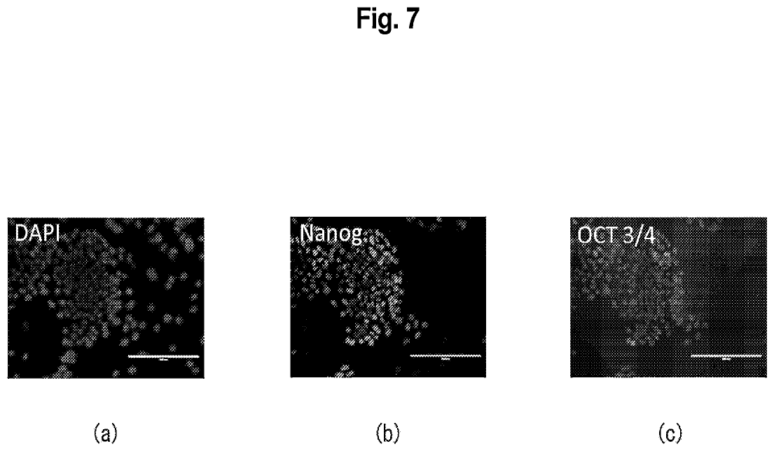

As shown in FIG. 5, when the cells that had been passaged only once were stained with anti-Sendai virus antibodies and the Sendai virus remaining in the cells was evaluated with a flow cytometer, the Sendai virus in the cells disappeared. As shown in FIG. 6, the Sendai virus remaining in the cells was not detected by PCR. As in the prior art, when cells were seeded at a high concentration at which 11 or more cells were adhered to each other, the Sendai virus remained in the cells. FIG. 7 shows immunostaining images of the obtained TRA1-60 positive cells.

### Example 5

SeV(PM)hKOS/TS12ΔF and SeV(HNL)hC-Myc/TS15ΔF were added to two-dimensionally cultured mononuclear cells so that the MOI was 5, the dish for inducing pluripotent stem cells was accommodated in an incubator at 34° C., and the cells were cultured. Two days after infection, the blood medium was replaced with an iPS cell medium. Then, the medium was replaced once every two days using the iPS cell medium. Along the way, the temperature was raised to 38° C.

8 days after infection, stem-cell-like cell masses generated. On the 14th day after infection, almost all cells became TRA1-60 positive cells, and showed iPS-cell-like morphology. 15 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Next, the well-dish was accommodated in an incubator at 38° C., and the cells were two-dimensionally cultured. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other.

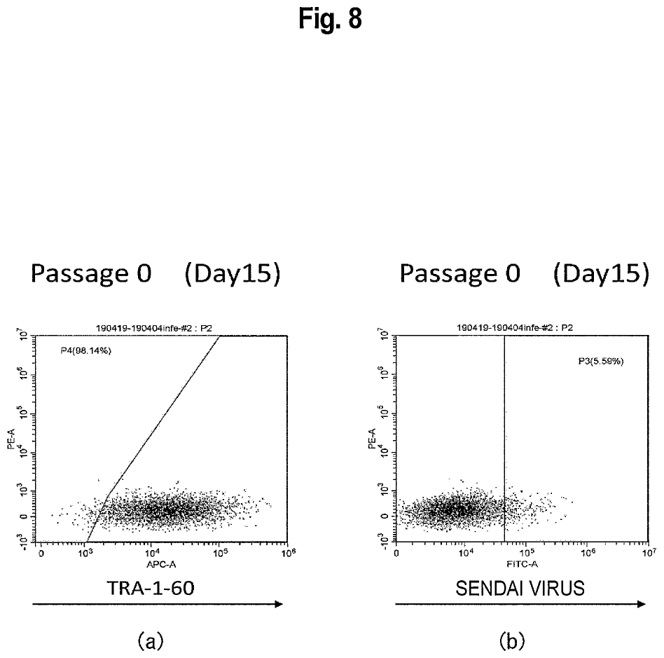

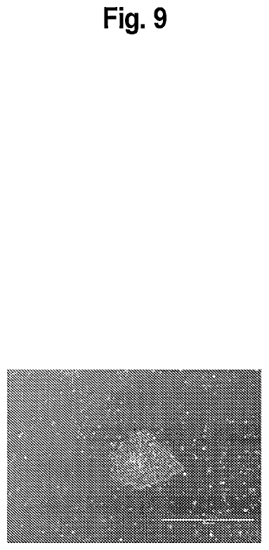

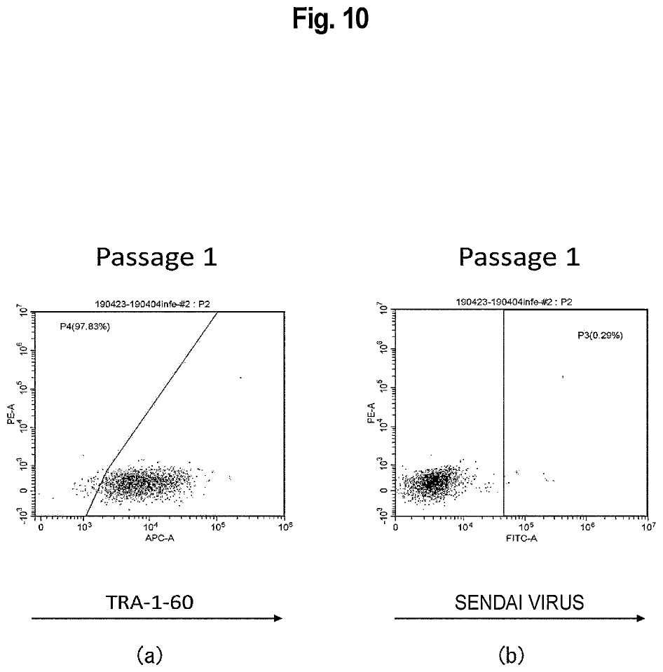

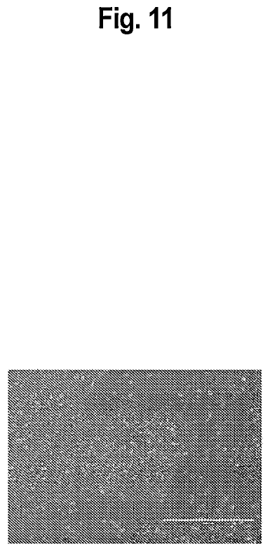

Using anti-Sendai virus antibodies, 15 days after infection, cells before passage were stained, the Sendai virus remaining in the cells was evaluated with a flow cytometer, and the results are shown in FIG. 8. In addition, FIG. 9 shows an image of the cells 15 days after infection. The cells that were passaged once were stained using anti-Sendai virus antibodies, the Sendai virus remaining in the cells was evaluated with a flow cytometer, and the results are shown in FIG. 10. As shown in FIG. 10, in the first passage, the Sendai virus in the cells almost disappeared. FIG. 11 shows an image of the cells in the first passage.

### Example 6

SeV(PM)hKOS/TS12ΔF, SeV18+hKLF4/TSΔF, and SeV(HNL)hC-Myc/TS15ΔF were added to mononuclear cells that were three-dimensionally cultured in a polymer-containing blood medium so that the MOI was 5, a dish for inducing pluripotent stem cells was accommodated in an incubator at 37° C. and the cells were cultured. Two days after infection, the polymer-containing blood medium was replaced with a polymer-containing iPS cell medium. Then, the medium was replaced once every two days using the polymer-containing iPS cell medium.

14 days after infection, stem-cell-like cell masses generated. On the 14th day after infection, almost all cells were TRA1-60 positive cells. Here, when some of the obtained TRA1-60 positive cells were seeded and two-dimensionally cultured in an incubator, iPS-cell-like colonies were formed. In addition, the cell masses were recovered using a mesh, a TrypLE select as a cell-releasing agent was added to the recovered cells, the cells were left at room temperature for 5 minutes, the cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Then, the well-dish was accommodated in an incubator at 37° C., and cells were two-dimensionally cultured. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other. Along the way, the temperature was raised to 38° C.

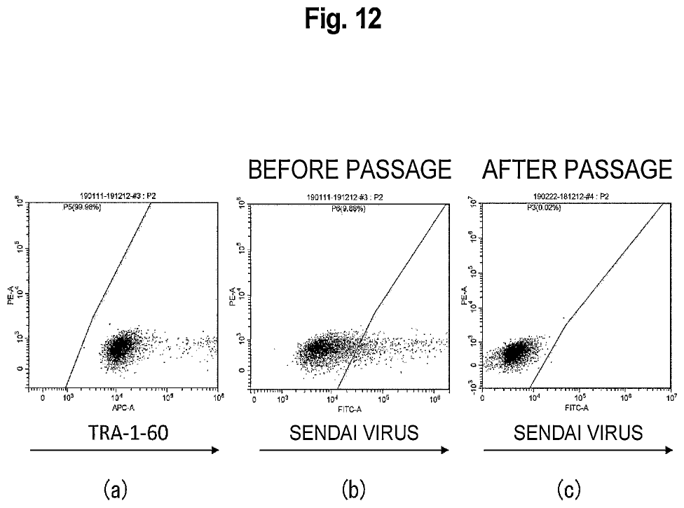

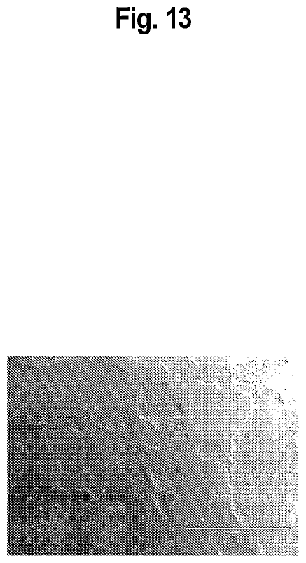

When the cells that were passaged twice were stained using anti-Sendai virus antibodies, and the Sendai virus remaining in the cells was evaluated with a flow cytometer, as shown in FIG. 12, the Sendai virus in the cells almost disappeared. FIG. 13 shows an image of the cells that were passaged twice.

### Example 7

SeV(PM)hKOS/TS12ΔF and SeV(HNL)hC-Myc/TS15ΔF were added to two-dimensionally cultured fibroblasts so that the MOI was 5, a dish for inducing pluripotent stem cells was accommodated in an incubator at 34° C., and the cells were cultured. Two days after infection, the blood medium was replaced with an iPS cell medium. Then, the medium was replaced once every two days using the iPS cell medium.

8 days after infection, stem-cell-like cell masses generated. On the 14th day after infection, almost all cells became TRA1-60 positive cells, and showed iPS-cell-like morphology. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, the cells were seeded in the well plate so that the concentration was 0.25×10⁴ cells/cm² or less, and the first passage was performed. In this case, 11 or more cells did not come into contact with each other. Then, the well-dish was accommodated in an incubator at 37° C., and cells were two-dimensionally cultured. Then, the cells were passaged so that the cells had 60% to 80% confluency. From the second passage onward, the cells were seeded in the well plate so that the concentration was 0.25×10⁴ cells/cm² or less. In this case also, 11 or more cells did not come into contact with each other.

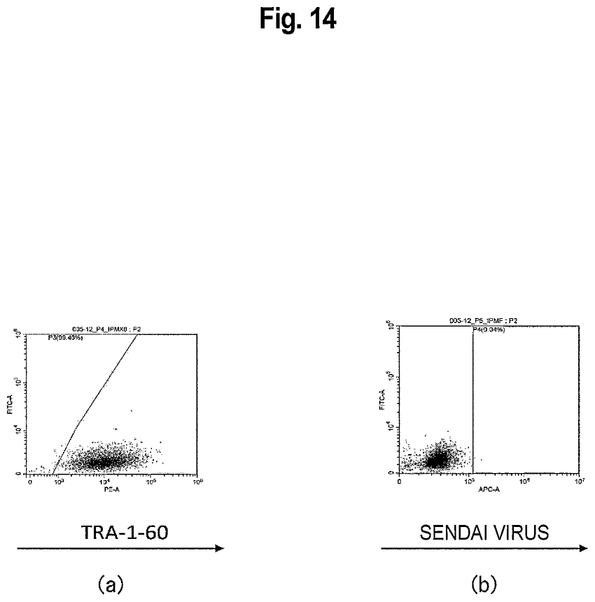

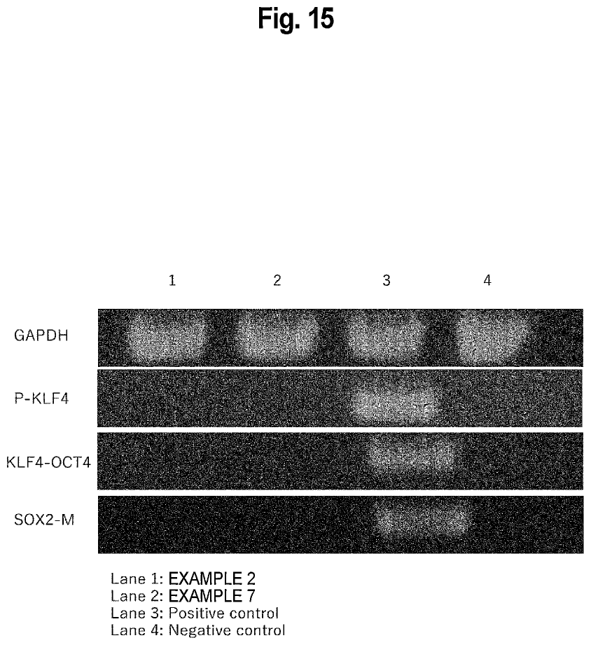

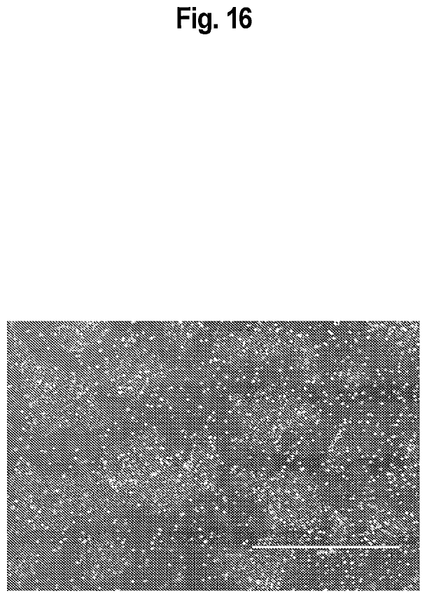

As shown in FIG. 14, when the cells were stained using anti-Sendai virus antibodies and the Sendai virus remaining in the cells was evaluated with a flow cytometer, the Sendai virus in the cells disappeared after the first passage. As shown in FIG. 15, the Sendai virus remaining in the cells was not detected by PCR. FIG. 16 shows an image of the obtained TRA1-60 positive cells.

### Comparative Example 1

SeV(PM)hKOS/TS12ΔF, SeV18+hKLF4/TSΔF, and SeV(HNL)hC-Myc/TS15ΔF were added to two-dimensionally cultured fibroblasts so that the MOI was 5, a dish for inducing pluripotent stem cells was accommodated in an incubator at 37° C. and the cells were cultured. Two days after infection, the blood medium was replaced with an iPS cell medium. Then, the medium was replaced once every two days using the iPS cell medium. On the 14th day after infection, the culture temperature was raised to 37° C.

10 days after infection, stem-cell-like cell masses generated. 14 days after infection, a TrypLE select as a cell-releasing agent was added to the dish, and a cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, cells were seeded in a well plate at a high concentration at which 11 or more cells were adhered to each other, and the first passage was performed. Next, the well-dish was accommodated in an incubator, and the cells were two-dimensionally culture. Then, the cells were passaged so that the cells had 60% to 80% confluency. From the second passage onward, the cells were seeded in the well plate at a high concentration at which 11 or more cells were adhered to each other.

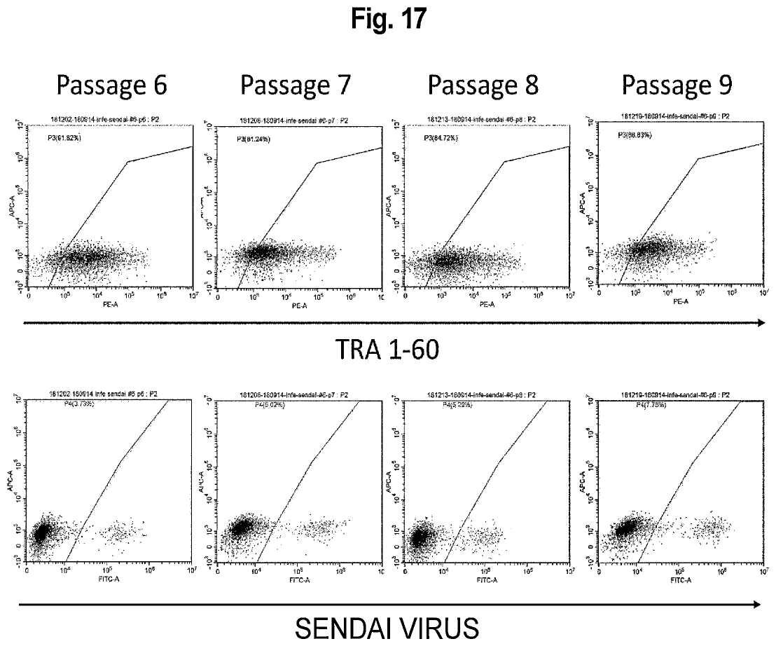

When the cells were stained using anti-Sendai virus antibodies and the Sendai virus remaining in the cells was evaluated with a flow cytometer, as shown in FIG. 17, even after passage was repeated, the Sendai virus continued to remain in the cells.

### Comparative Example 2

SeV(PM)hKOS/TS12ΔF, SeV18+hKLF4/TSΔF, and SeV(HNL)hC-Myc/TS15ΔF were added to mononuclear cells that were three-dimensionally cultured in a polymer-containing blood medium so that the MOI was 5, a dish for inducing pluripotent stem cells was accommodated in an incubator at 37° C. and the cells were cultured. Two days after infection, the polymer-containing blood medium was replaced with a polymer-containing iPS cell medium. Then, the medium was replaced once every two days using the polymer-containing iPS cell medium.

10 days after infection, stem-cell-like cell masses generated. 14 days after infection, the cell masses were recovered using a mesh, a TrypLE select as a cell-releasing agent was added to the recovered cells, the cells were left at room temperature for 5 minutes, the cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, cells were seeded in a well plate at a high concentration at which 11 or more cells were adhered to each other, and the first passage was performed. Next, the well-dish was accommodated in an incubator at 37° C., and cells were two-dimensionally cultured. Then, the cells were passaged so that the cells had 60% to 80% confluency. From the second passage onward, the cells were seeded in the well plate at a high concentration at which 11 or more cells were adhered to each other. Along the way, the temperature was raised to 38° C.

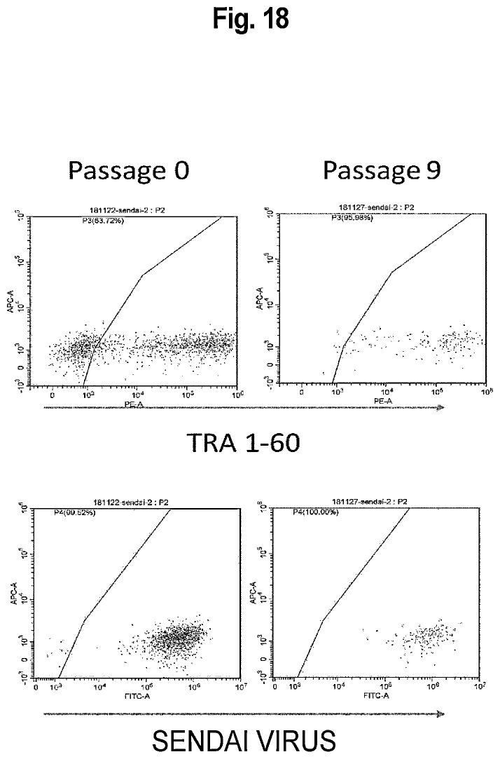

When the cells were stained using anti-Sendai virus antibodies and the Sendai virus remaining in the cells was evaluated with a flow cytometer, as shown in FIG. 18, even after the 9th passage, the Sendai virus continued to remain in the cells. After infection, the dish for inducing pluripotent stem cells was accommodated in an incubator at 34° C., and even when other conditions were the same and pluripotent stem cells were induced, the Sendai virus continued to remain in the cells.

### Comparative Example 3

SeV(PM)hKOS/TS12ΔF, SeV18+hKLF4/TSΔF, and SeV(HNL)hC-Myc/TS15ΔF were added to mononuclear cells that were three-dimensionally cultured in a polymer-containing blood medium so that the MOI was 5, a dish for inducing pluripotent stem cells was accommodated in an incubator at 34° C., and the cells were cultured. Two days after infection, the polymer-containing blood medium was replaced with a polymer-containing iPS cell medium. Then, the medium was replaced once every two days using the polymer-containing iPS cell medium. Along the way, the temperature was raised to 37° C. and additionally raised to 38° C.

10 days after infection, stem-cell-like cell masses generated. 14 days after infection, the cell masses were recovered using a mesh, a TrypLE select as a cell-releasing agent was added to the recovered cells, the cells were left at room temperature for 5 minutes, the cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, cells were seeded in a well plate at a high concentration at which 11 or more cells were adhered to each other, and the first passage was performed. Next, the well-dish was accommodated in an incubator at 37° C., and cells were two-dimensionally cultured. After the cells began to divide, the culture temperature was raised to 38° C. Then, the cells were passaged so that the cells had 60% to 80% confluency. From the second passage onward, the cells were seeded in the well plate at a high concentration at which 11 or more cells were adhered to each other. Along the way, the temperature was raised to 38° C.

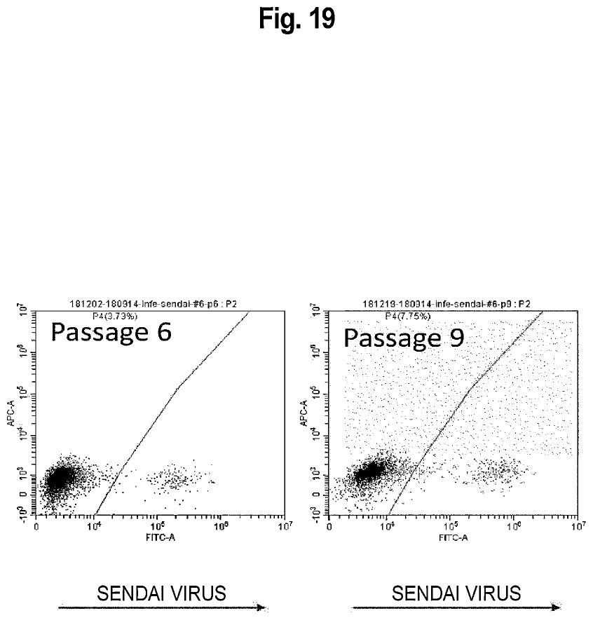

When the cells were stained using anti-Sendai virus antibodies and the Sendai virus remaining in the cells was evaluated with a flow cytometer, as shown in FIG. 19, even after passage was repeated, the Sendai virus continued to remain in the cells.

### Comparative Example 4

SeV(PM)hKOS/TS12ΔF and SeV(HNL)hC-Myc/TS15ΔF were added to two-dimensionally cultured mononuclear cells so that the MOI was 5, the dish for inducing pluripotent stem cells was accommodated in an incubator at 34° C., and the cells were cultured. Two days after infection, the blood medium was replaced with an iPS cell medium. Then, the medium was replaced once every two days using the iPS cell medium. Along the way, the temperature was raised to 37° C. and the temperature was additionally raised to 38° C.

8 days after infection, stem-cell-like cell masses generated. 14 days after infection, a TrypLE select as a cell-releasing agent was added to the dish and left at room temperature for 1 minute, and a cell-containing solution was then sucked up, and the cell-containing solution was incubated at 37° C. for 5 minutes to 10 minutes. Then, an iPS cell medium was added, and the cell-containing iPS cell medium was recovered in a 15 mL tube. The number of cells was measured using a blood cell counting chamber, the concentration of the cell-containing solution was adjusted, cells were seeded in a well plate at a high concentration at which 11 or more cells were adhered to each other, and the first passage was performed. Next, the well-dish was accommodated in an incubator at 38° C., and the cells were two-dimensionally cultured. Then, the cells were passaged so that the cells had 60% to 80 confluency. From the second passage onward, the cells were seeded in the well plate at a high concentration at which 11 or more cells were adhered to each other.

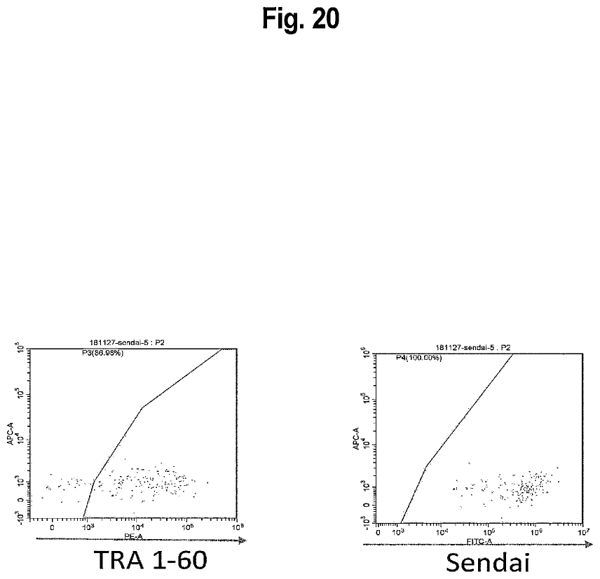

When the cells that had been passaged 9 times were stained with anti-Sendai virus antibodies and the Sendai virus remaining in the cells was evaluated with a flow cytometer, as shown in FIG. 20, the Sendai virus remained in the cells.

### Example 8

A DMEM containing 10% FBS was prepared as a medium for fibroblasts. Fibroblasts were suspended in a medium for adult human-derived fibroblasts to obtain a fibroblast suspension.

A solution in which 1.5 mL of PBS and 4.8 μL of silkworm-derived laminin (iMatrix-511 silk, nippi) were mixed was added to one well of a 6-well dish, and the dish was left in an incubator at 37° C. for 1 hour. Next, a solution in which PBS and laminin were mixed was removed from the well using an aspirator, and 1.5 mL of the fibroblast suspension was added to one well. The number of fibroblasts in one well was 0.5×10⁵ to 2.0×10⁵. Then, the fibroblasts were cultured in an incubator at 37° C. for 1 day.

Next, the medium was replaced with a stem cell induction medium. The amount of the medium replaced was 1.5 mL.

A tube A and a tube B were prepared, and 0.1 μL to 100 μL of a mixture containing OCT4 RNA, SOX2 RNA, KLF4 RNA, and C-MYC RNA (100 ng/μL) was added to 125 μL of PBS in the tube A. 0.1 μL to 100 μL of a lipofection reagent was added to 125 μL of PBS in the tube B. Next, the solution in the tube A and the solution in the tube B were mixed, the mixed solution was left at room temperature for 10 minutes, and a total amount of the mixed solution was added to the medium in one well. Then, the dish was left in an incubator at 37° C. for 1 day, and the cells were transfected with RNA. Then, RNA transfection was repeated 11 times according to the same procedures.

The day after the 11th RNA transfection, the concentration of the cell-containing solution was adjusted, and the cells were seeded in a laminin-coated well plate for the first passage so that the concentration was 0.25×10⁴ cells/cm² or less. In this case, 11 or more cells did not come into contact with each other. Next, the well-dish was accommodated in an incubator at 37° C., and cells were two-dimensionally cultured. Then, the cells were passaged only once when the cells became 60% to 80% confluency. From the second passage onward, the cells were seeded in the well plate so that the concentration was 0.25×10⁴ cells/cm² or less. In this case, 11 or more cells did not come into contact with each other.

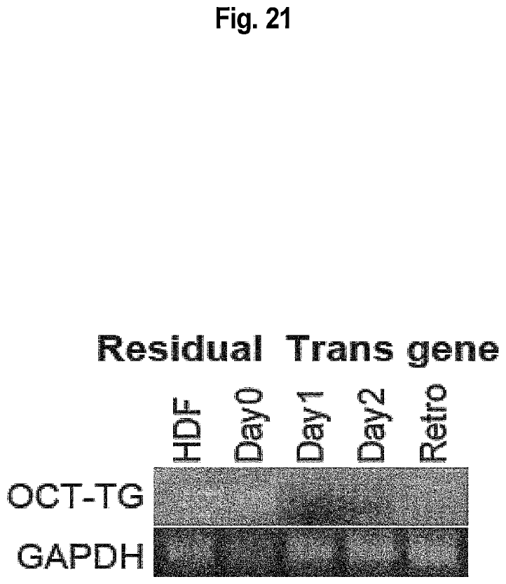

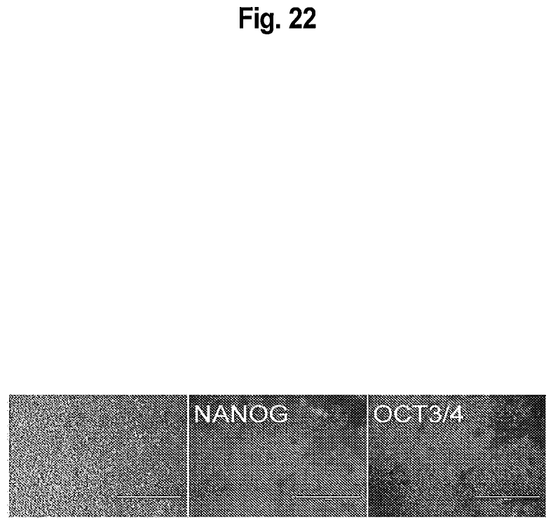

As shown in FIG. 21, on the first day after the first passage, the reprogramming factor almost disappeared from the cells, and on the second day after the first passage, the reprogramming factor completely disappeared from the cells. On the 10th day from infection, almost all cells became TRA1-60 positive cells, and showed iPS-cell-like morphology. FIG. 22 shows an immunostaining image of the TRA1-60 positive cells.

### Example 9

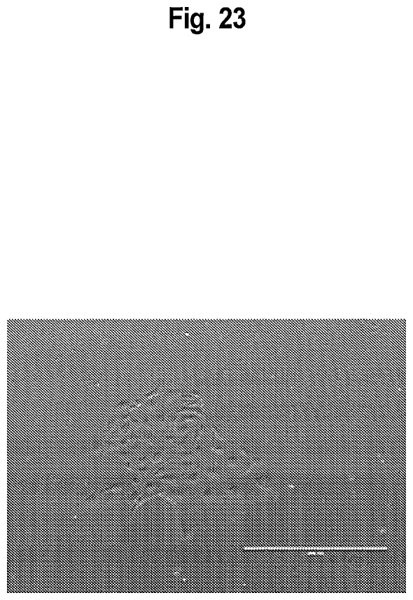

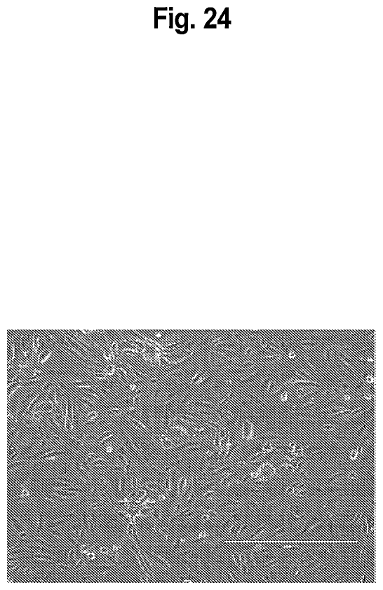

300 mL of urine was collected from a healthy subject, 6 urine samples were dispensed into a 50 mL Falcon tube, and the tube was centrifuged at 400G for 5 minutes. After centrifugation, the supernatant was removed from the tube, 30 mL of PBS was put into the tube, and the tube was centrifuged at 400G for 5 minutes. After centrifugation, the supernatant was removed from the tube, 30 mL of a primary medium was put into the tube, and the tube was centrifuged at 400G for 5 minutes. A primary medium was prepared by adding fetal bovine serum (Gibco, 10437028, final concentration 10%), SingleQuots Kit CC-4127 REGM (Lonza, 1/1000 amount), and Antibiotic-Antimycotic (Gibco, 15240062, 1/100 amount) to DMEM/Ham's F12 (Gibco, 11320-033). After centrifugation, the supernatant was removed from the tube, cells were suspended in 1 mL of the primary medium, the cells were seeded in one well of a gelatin-coated 24-well plate, and the cells were incubated in an incubator at 37° C. For 2 days after the cells were seeded, the primary medium was added to a 300 μL well, and from the 3d day onward, the medium was replaced using a medium for epithelial cells. The medium for epithelial cells was prepared by adding SingleQuots Kit CC-4127 REGM (Lonza) to a renal epithelial cell basal medium (Lonza). FIG. 23 shows a microscope image of the cells after expansion-cultured for 6 days. The cells were subjected to the first passage on the 7th day after seeding, the cells were additionally expansion-cultured, and the cells were subjected to the second passage on the 7th day after the first passage. FIG. 24 shows a microscope image of the cells on the 6th day after the second passage.

### Example 10

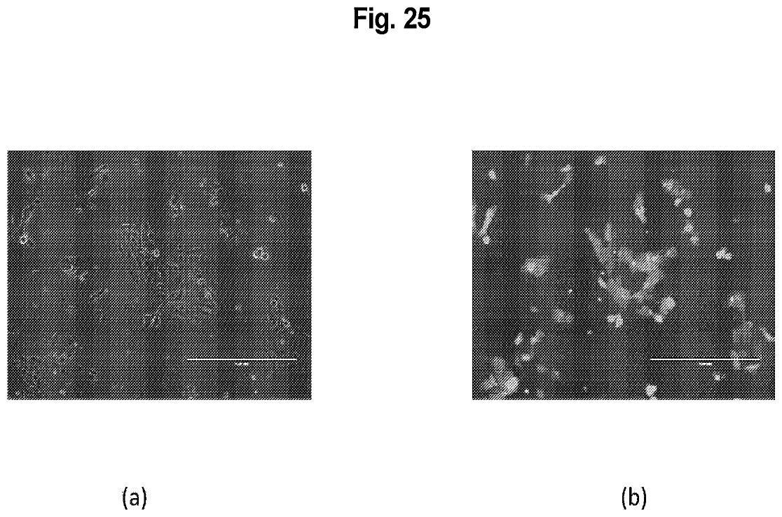

A dish coated with laminin 511 was used as a dish for inducing pluripotent stem cells. Urine-derived cells prepared in Example 9 were seeded at a low concentration in the dish for inducing pluripotent stem cells and incubated at 37° C. For the medium, a medium for epithelial cells was used. The next day, a mixture containing a transfection reagent and RNA that encodes a green fluorescent protein (GFP) was added to a medium, the medium was replaced with the above medium, and incubated at 37° C. FIG. 25 shows microscope images of the cells on the next day. Expression of GFP was observed, which indicates that transfection into urine-derived cells was performed.

### Example 11

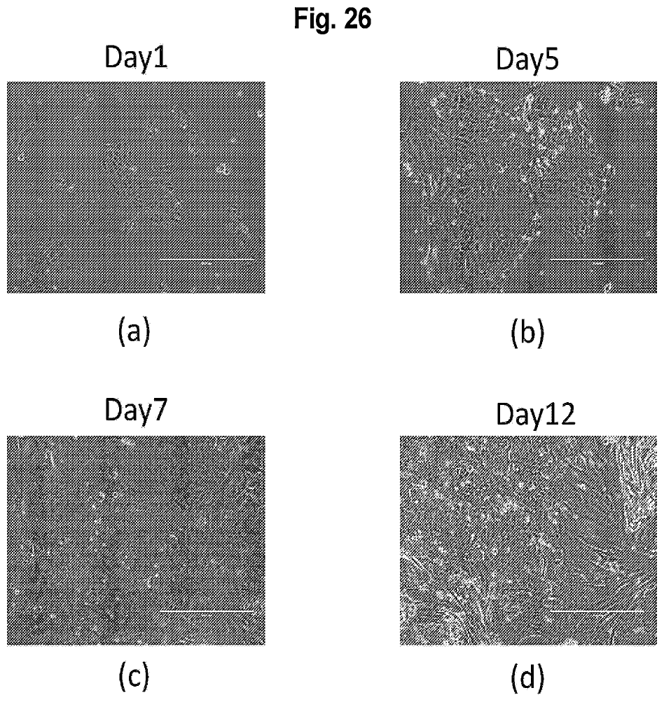

A dish coated with laminin 511 was used as a dish for inducing pluripotent stem cells. Urine-derived cells prepared in Example 9 were seeded at a low concentration in the dish for inducing pluripotent stem cells and incubated at 37° C. For the medium, a medium for epithelial cells was used. The next day, a tube A and a tube B were prepared, and 0.1 μL to 10² μL of a mixture containing M₃O RNA, SOX2 RNA, KLF4 RNA, C-MYC RNA, and LIN28 RNA (100 ng/μL) was added to 125 μL of PBS in the tube A. In addition, these RNAs were substantially concentrated and purified into single-stranded RNA through HPLC. It was confirmed that the ratio (A₂₆₀/A₂₈₀) of the absorbances of RNA at 260 nm and 280 nm was 1.71 to 2.1, and proteins were not substantially mixed. In addition, when dot blot analysis was performed using anti-double-stranded RNA antibody J2, 90% or more of double-stranded RNA was removed. 0.1 μL to 100 μL of a lipofection reagent was added to 125 μL of PBS in the tube B. Next, the solution in the tube A and the solution in the tube B were mixed, the mixed solution was left at room temperature for 10 minutes, a total amount of the mixed solution was added to a transfection medium without using B18R or the like, the medium was replaced using the transfection medium, and incubated at 37° C. Transfection was performed once daily for 10 days. Observation was performed on days 1, 5, 7, and 14 after cell seeding. As shown in FIG. 26, it was observed that cell morphology changed like ES cells as the day progressed.

### Example 12

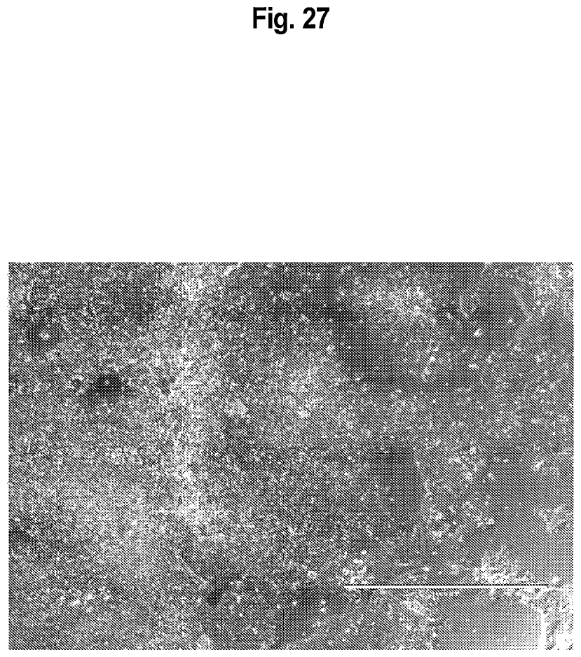

As in Example 11, urine-derived cells were transfected for 10 days. The cells were cultured in a medium for stem cells (StemFit, Ajinomoto) from the 11th day after the cells were seeded, all cells were separated from the dish on the 14th day, and some of the recovered and mixed cells were seeded and passaged in the medium. During passage, without picking up colonies, all cells on the dish were recovered, and 1×10² to 1×10⁵ cells were seeded on the dish. FIG. 27 shows a microscope image of the cells 6 days after passage. ES-cell-like cells were confirmed.

### Example 13

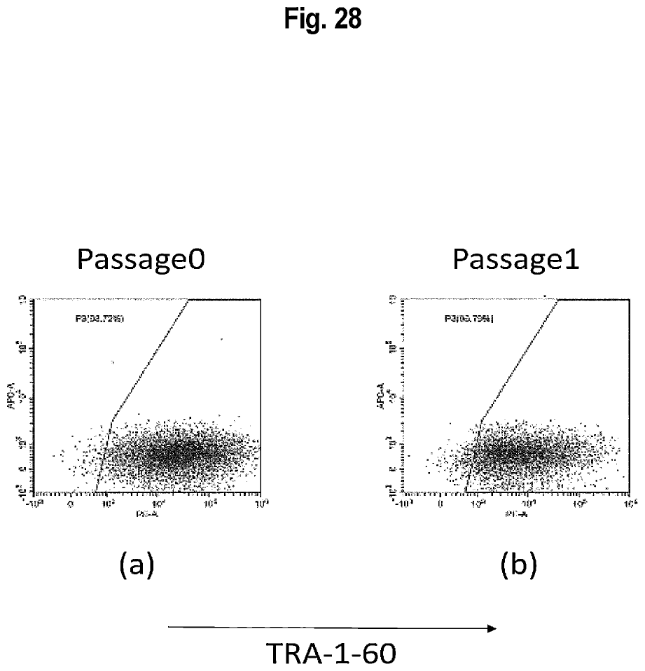

As in Example 11, urine-derived cells were transfected for 10 days. When the cells were cultured in a medium for stem cells (StemFit, Ajinomoto) from the 11th day after the cells were seeded, the cells were separated from the dish on the 14th day, and some of the cells were analyzed using a flow cytometry, as shown in FIG. 28(a), TRA-1-60 positive was confirmed. In addition, when the cells separated from the dish on the 14th day were passaged, and analyzed using a flow cytometry 7 days later, as shown in FIG. 28(b), TRA-1-60 positive was confirmed.

### Example 14

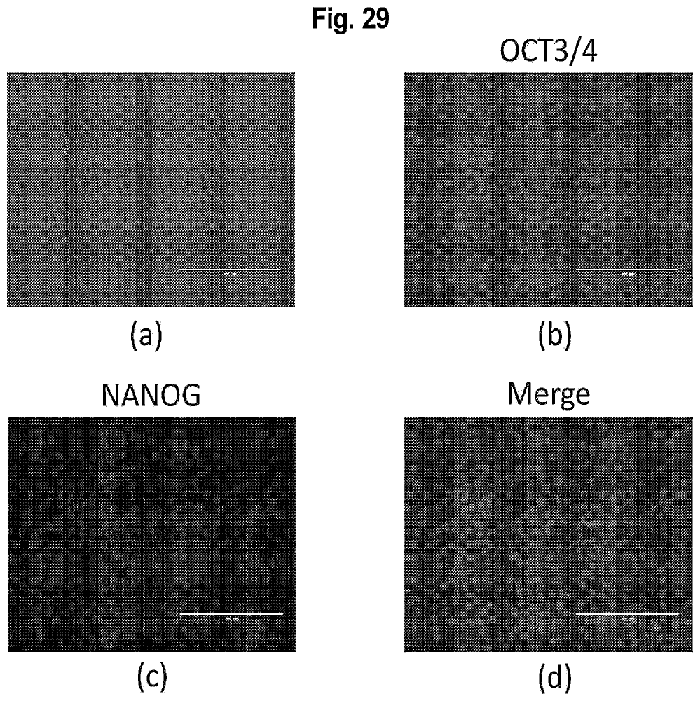

As in Example 11, urine-derived cells were transfected for 10 days. The cells were cultured in a medium for stem cells (StemFit, Ajinomoto) from the 11th day after the cells were seeded, all cells were separated from the dish on the 14th day, and some of the separated and mixed cells were seeded and passaged. StemFit (registered trademark) was used as the medium after passage. 7 days after passage, cells were immobilized, and the cells were stained using anti-OCT3/4 antibodies and anti-NANOG antibodies. In addition, nuclei chemical staining using Hoechst (registered trademark) was performed. As a result, as shown in FIG. 29, expression of OCT3/4 and NANOG, which are specific markers for pluripotent stem cells, in cell nuclei was confirmed. Therefore, it was shown that the pluripotent stem cells could be induced from urine-derived cells using RNA. Here, FIG. 29(d) is an image obtained by synthesizing an image of cells stained using anti-OCT3/4 antibodies, an image of cells stained using anti-NANOG antibodies, and an image of cells stained using Hoechst (registered trademark).

### Example 17

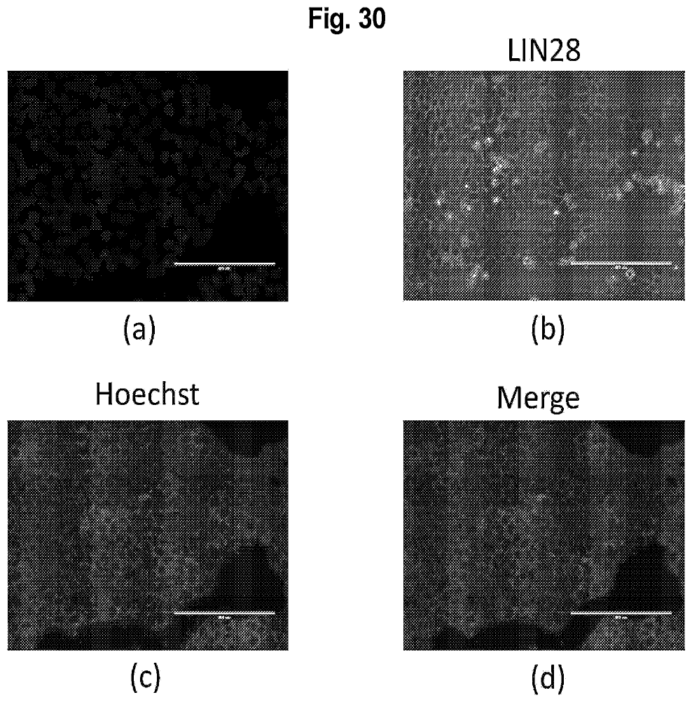

As in Example 11, urine-derived cells were transfected for 10 days. The cells were cultured in a medium for stem cells (StemFit, Ajinomoto) from the 11th day after the cells were seeded, all cells were separated from the dish using a TrypLE select on the 14th day, and some of the separated and mixed cells were seeded and passaged in a medium. StemFit (registered trademark) was used as the medium after passage. 7 days after passage, cells were immobilized, and the cells were stained using anti-LIN28 antibodies. In addition, nuclei chemical staining using Hoechst (registered trademark) was performed. As a result, as shown in FIG. 30, expression of LIN28, which is a specific marker for pluripotent stem cells, in cell nuclei was confirmed. Therefore, it was shown that the pluripotent stem cells could be induced from urine-derived cells using RNA. Here, FIG. 30(d) is an image obtained by synthesizing an image of cells stained using anti-LIN28 antibodies and an image of cells stained using Hoechst (registered trademark).

1. A method for manufacturing pluripotent stem cells, the method comprising:
introducing a reprogramming factor into cells; and
seeding the cells, into which the reprogramming factor is introduced, at a low concentration and culturing the cells.

2. The method according to claim 1,
wherein the low concentration is 0.25×10⁴ cells/cm² or less.

3. The method according to claim 1,
wherein the low concentration is a concentration at which 11 or more of the seeded cells do not come into contact with each other.

4. The method according to claim 1,
wherein the low concentration is 5% or less confluency.

5. The method according to claim 1,
wherein the reprogramming factor is RNA.

6. The method according to claim 1,
wherein the reprogramming factor is introduced into the cells by a lipofection method.

7. The method according to claim 1,
wherein the reprogramming factor is introduced into the cells by using a viral vector.

8. The method according to claim 7,
wherein the viral vector is a temperature-sensitive viral vector in which stability of a viral nucleic acid decreases at a predetermined temperature or higher.

9. The method according to claim 8,
wherein the viral vector does not include a viral vector in which the stability of a viral nucleic acid does not decrease at the predetermined temperature or higher.

10. The method according to claim 8,
wherein, after the reprogramming factor is introduced, the cells are cultured at a temperature lower than the predetermined temperature for at least two days.

11. The method according to claim 8,
wherein, in the culturing, the cells into which the reprogramming factor is introduced are passaged and cultured at the predetermined temperature or higher.

12. The method according to claim 1,
wherein, in the culturing, pluripotent stem cells or colonies of pluripotent stem cells are not isolated.

13. The method according to claim 1,
wherein, in the culturing, the cells into which the reprogramming factor is introduced are not cloned.

14. The method according to claim 1,
wherein, in the culturing, the cells into which the reprogramming factor is introduced are separated from an incubator, and at least some of the separated cells are mixed and seeded.

15. The method according to claim 1,
wherein, in the culturing, the cells into which the reprogramming factor is introduced are recovered from an incubator, and at least some of the recovered cells are mixed and seeded.

16. The method according to claim 1,
wherein, in the culturing, each of a plurality of colonies formed by the cells into which the reprogramming factor is introduced is not picked up.

17. The method according to claim 1,
wherein, in the culturing, cells derived from different single cells, which are the cells into which the reprogramming factor is introduced, are mixed and seeded.

18. The method according to claim 1,
wherein, after the culturing, all cells are cryopreserved as the pluripotent stem cells.

19. The method according to claim 1,
wherein, after the culturing, all separated cells are cryopreserved as the pluripotent stem cells.

20. The method according to claim 1,
wherein the culturing is adherent culture.

21. The method according to claim 1,
wherein the culturing is suspension-culture.

22. The method according to claim 1,
wherein, after the culturing, colonies of pluripotent stem cells are mixed with each other.

23. The method according to claim 1,
wherein the cells into which the reprogramming factor is introduced are derived from blood cells, fibroblasts, or cells contained in urine.

24.-26. (canceled)
